SEQUENCE LISTING (90 sequences)

SEQ ID NO: 1 - protein, 116 aa, Artificial Sequence
  Synthetic Sequence
  MISC_FEATURE (4)..(4): Xaa1 is leucine (Leu) or phenylalanine (Phe)
  MISC_FEATURE (5)..(5): Xaa2 is valine (Val), methionine (Met), or leucine (Leu)
  MISC_FEATURE (44)..(44): Xaa3 is arginine (Arg) or glycine (Gly)
  MISC_FEATURE (56)..(56): Xaa4 is glycine (Gly), serine (Ser), or alanine (Ala)
  MISC_FEATURE (72)..(72): Xaa5 is arginine (Arg) or alanine (Ala)
  MISC_FEATURE (74)..(74): Xaa6 is threonine (Thr) or lysine (Lys)
  MISC_FEATURE (84)..(84): Xaa7 is serine (Ser) or alanine (Ala)
  MISC_FEATURE (95)..(95): Xaa8 is tyrosine (Tyr) or phenylalanine (Phe)
QVQXXQSGAEVKKPGASVKVSCKASGFTFTSYDINWVRQAPGQXLEWMGWIYPGDXSTKYNEKFKGRVTITXDXSASTAYMELXSLRSEDTAVYXCTRSFYTMDYWGQGTTVTVSS

SEQ ID NO: 2 - protein, 116 aa, Artificial Sequence
  Synthetic Sequence
QVQLVQSGAEVKKPGASVKVSCKASGFTFTSYDINWVRQAPGQRLEWMGWIYPGDGSTKYNEKFKGRVTITRDTSASTAYMELSSLRSEDTAVYYCTRSFYTMDYWGQGTTVTVSS

SEQ ID NO: 3 - protein, 116 aa, Artificial Sequence
  Synthetic Sequence
QVQLVQSGAEVKKPGASVKVSCKASGFTFTSYDINWVRQAPGQGLEWMGWIYPGDGSTKYNEKFKGRVTITADKSASTAYMELSSLRSEDTAVYFCTRSFYTMDYWGQGTTVTVSS

SEQ ID NO: 4 - protein, 116 aa, Artificial Sequence
  Synthetic Sequence
QVQLMQSGAEVKKPGASVKVSCKASGFTFTSYDINWVRQAPGQRLEWMGWIYPGDSSTKYNEKFKGRVTITRDKSANTAYMELASLRSEDTAVYFCTRSFYTMDYWGQGTTVTVSS

SEQ ID NO: 5 - protein, 116 aa, Artificial Sequence
  Synthetic Sequence
QVQLMQSGAEVKKPGASVKVSCKASGFTFTSYDINWVRQAPGQRLEWMGWIYPGDASTKYNEKFKGRVTITRDKSANTAYMELASLRSEDTAVYFCTRSFYTMDYWGQGTTVTVSS

SEQ ID NO: 6 - protein, 116 aa, Artificial Sequence
  Synthetic Sequence
QVQLMQSGAEVKKPGASVKVSCKASGFTFTSYDINWVRQAPGQRLEWMGWIYPGDSSTKYNEKFKGRVTITADKSANTAYMELASLRSEDTAVYFCTRSFYTMDYWGQGTTVTVSS

SEQ ID NO: 7 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITRDKSANTAYMELSSLRSEDTAVYFCTRSFYTMDYWGQGTTVTVSS

SEQ ID NO: 8 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITADKSANTAYMELASLRSEDTAVYFCTRSFYTMDYWGQGTTVTVSS

SEQ ID NO: 9 - protein, 116 aa, Artificial Sequence
  Synthetic Sequence
QVQLLQSGAEVKKPGASVKVSCKASGFTFTSYDINWVRQAPGQRLEWMGWIYPGDASTKYNEKFKGRVTITRDKSANTAYMELASLRSEDTAVYFCTRSFYTMDYWGQGTTVTVSS

SEQ ID NO: 10 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITRDKSANTAYMELASLRSEDTAVYFCTRSFYTMDYWGQGTTVTVSS

SEQ ID NO: 11 - protein, 116 aa, Artificial Sequence
  Synthetic Sequence
QVQFLQSGAEVKKPGASVKVSCKASGFTFTSYDINWVRQAPGQRLEWMGWIYPGDSSTKYNEKFKGRVTITADKSANTAYMELSSLRSEDTAVYFCTRSFYTMDYWGQGTTVTVSS

SEQ ID NO: 12 - protein, 116 aa, Artificial Sequence
  Synthetic Sequence
QVQFVQSGAEVKKPGASVKVSCKASGFTFTSYDINWVRQAPGQRLEWMGWIYPGDSSTKYNEKFKGRVTITRDKSANTAYMELSSLRSEDTAVYFCTRSFYTMDYWGQGTTVTVSS

SEQ ID NO: 13 - protein, 116 aa, Artificial Sequence
  Synthetic Sequence
QVQFVQSGAEVKKPGASVKVSCKASGFTFTSYDINWVRQAPGQRLEWMGWIYPGDSSTKYNEKFKGRVTITADKSANTAYMELSSLRSEDTAVYFCTRSFYTMDYWGQGTTVTVSS

SEQ ID NO: 14 - protein, 116 aa, Artificial Sequence
  Synthetic Sequence
QVQLLQSGAEVKKPGASVKVSCKASGFTFTSYDINWVRQAPGQRLEWMGWIYPGDSSTKYNEKFKGRVTITRDKSANTAYMELSSLRSEDTAVYYCTRSFYTMDYWGQGTTVTVSS

SEQ ID NO: 15 - protein, 120 aa, Artificial Sequence
  Synthetic Sequence
  MISC_FEATURE (33)..(33): Xaa1 is tryptophan (Trp) or tyrosine (Tyr)
  MISC_FEATURE (35)..(35): Xaa2 is histidine (His), asparagine (Asn), or tyrosine (Tyr)
  MISC_FEATURE (42)..(42): Xaa3 is glycine (Gly) or arginine (Arg)
  MISC_FEATURE (52)..(52): Xaa4 is aspartic acid (Asp), glutamic acid (Glu), or histidine (His)
  MISC_FEATURE (54)..(54): Xaa5 is serine (Ser), threonine (Thr), or tyrosine (Tyr)
  MISC_FEATURE (55)..(55): Xaa6 is asparagine (Asn) or glycine (Gly)
  MISC_FEATURE (56)..(56): Xaa7 is serine (Ser), alanine (Ala), or aspartic acid (Asp)
QVQLVQSGAEVKKPGASVKVSCKASGYTFTNYXMXWVRQAPXQGLEWMGMFXPXXXVTRLNQKFKDRVTMTRDTSTSTVYMELSSLRSEDTAVYYCARTTSMIIGGFAYWGQGTLVTVSS

SEQ ID NO: 16 - protein, 120 aa, Artificial Sequence
  Synthetic Sequence
QVQLVQSGAEVKKPGASVKVSCKASGYTFTNYWMHWVRQAPGQGLEWMGMFDPSNSVTRLNQKFKDRVTMTRDTSTSTVYMELSSLRSEDTAVYYCARTTSMIIGGFAYWGQGTLVTVSS

SEQ ID NO: 17 - protein, 120 aa, Artificial Sequence
  Synthetic Sequence
QVQLVQSGAEVKKPGASVKVSCKASGYTFTNYWMHWVRQAPGQGLEWMGMFEPSNAVTRLNQKFKDRVTMTRDTSTSTVYMELSSLRSEDTAVYYCARTTSMIIGGFAYWGQGTLVTVSS

SEQ ID NO: 18 - protein, 120 aa, Artificial Sequence
  Synthetic Sequence
QVQLVQSGAEVKKPGASVKVSCKASGYTFTNYWMNWVRQAPRQGLEWMGMFEPSNAVTRLNQKFKDRVTMTRDTSTSTVYMELSSLRSEDTAVYYCARTTSMIIGGFAYWGQGTLVTVSS

SEQ ID NO: 19 - protein, 120 aa, Artificial Sequence
  Synthetic Sequence
QVQLVQSGAEVKKPGASVKVSCKASGYTFTNYWMYWVRQAPRQGLEWMGMFHPSNAVTRLNQKFKDRVTMTRDTSTSTVYMELSSLRSEDTAVYYCARTTSMIIGGFAYWGQGTLVTVSS

SEQ ID NO: 20 - protein, 120 aa, Artificial Sequence
  Synthetic Sequence
QVQLVQSGAEVKKPGASVKVSCKASGYTFTNYWMNWVRQAPRQGLEWMGMFHPSNAVTRLNQKFKDRVTMTRDTSTSTVYMELSSLRSEDTAVYYCARTTSMIIGGFAYWGQGTLVTVSS

SEQ ID NO: 21 - protein, 120 aa, Artificial Sequence
  Synthetic Sequence
QVQLVQSGAEVKKPGASVKVSCKASGYTFTNYYMNWVRQAPRQGLEWMGMFHPTGDVTRLNQKFKDRVTMTRDTSTSTVYMELSSLRSEDTAVYYCARTTSMIIGGFAYWGQGTLVTVSS

SEQ ID NO: 22 - protein, 120 aa, Artificial Sequence
  Synthetic Sequence
QVQLVQSGAEVKKPGASVKVSCKASGYTFTNYWMNWVRQAPRQGLEWMGMFHPTGDVTRLNQKFKDRVTMTRDTSTSTVYMELSSLRSEDTAVYYCARTTSMIIGGFAYWGQGTLVTVSS

SEQ ID NO: 23 - protein, 120 aa, Artificial Sequence
  Synthetic Sequence
QVQLVQSGAEVKKPGASVKVSCKASGYTFTNYYMNWVRQAPRQGLEWMGMFHPYGDVTRLNQKFKDRVTMTRDTSTSTVYMELSSLRSEDTAVYYCARTTSMIIGGFAYWGQGTLVTVSS

SEQ ID NO: 24 - protein, 120 aa, Artificial Sequence
  Synthetic Sequence
QVQLVQSGAEVKKPGASVKVSCKASGYTFTNYWMNWVRQAPRQGLEWMGMFHPYGDVTRLNQKFKDRVTMTRDTSTSTVYMELSSLRSEDTAVYYCARTTSMIIGGFAYWGQGTLVTVSS

SEQ ID NO: 25 - protein, 117 aa, Artificial Sequence
  Synthetic Sequence
  MISC_FEATURE (1)..(1): Xaa1 is glutamine (Gln) or aspartic acid (Asp)
  MISC_FEATURE (4)..(4): Xaa2 is leucine (Leu) or phenylalanine (Phe)
  MISC_FEATURE (25)..(25): Xaa3 is threonine (Thr) or serine (Ser)
  MISC_FEATURE (41)..(41): Xaa4 is proline (Pro) or phenylalanine (Phe)
  MISC_FEATURE (44)..(44): Xaa5 is lysine (Lys) or asparagine (Asn)
  MISC_FEATURE (45)..(45): Xaa6 is glycine (Gly) or lysine (Lys)
  MISC_FEATURE (71)..(71): Xaa7 is serine (Ser) or threonine (Thr)
  MISC_FEATURE (72)..(72): Xaa8 is valine (Val) or arginine (Arg)
  MISC_FEATURE (95)..(95): Xaa9 is tyrosine (Tyr) or phenylalanine (Phe)
XVQXQESGPGLVKPSQTLSLTCTVXGYSITSDFAWNWIRQXPGXXLEWIGYISYSGDTNYNPSLKSRVTIXXDTSKNQFSLKLSSVTAADTAVYXCAIRGPYSFTYWGQGTLVTVSS

SEQ ID NO: 26 - protein, 117 aa, Artificial Sequence
  Synthetic Sequence
QVQLQESGPGLVKPSQTLSLTCTVSGYSITSDFAWNWIRQPPGKGLEWIGYISYSGDTNYNPSLKSRVTISVDTSKNQFSLKLSSVTAADTAVYYCAIRGPYSFTYWGQGTLVTVSS

SEQ ID NO: 27 - protein, 117 aa, Artificial Sequence
  Synthetic Sequence
QVQFQESGPGLVKPSQTLSLTCTVSGYSITSDFAWNWIRQPPGKGLEWIGYISYSGDTNYNPSLKSRVTISRDTSKNQFSLKLSSVTAADTAVYFCAIRGPYSFTYWGQGTLVTVSS

SEQ ID NO: 28 - protein, 117 aa, Artificial Sequence
  Synthetic Sequence
QVQLQESGPGLVKPSQTLSLTCTVSGYSITSDFAWNWIRQFPGNKLEWIGYISYSGDTNYNPSLKSRVTISVDTSKNQFSLKLSSVTAADTAVYYCAIRGPYSFTYWGQGTLVTVSS

SEQ ID NO: 29 - protein, 117 aa, Artificial Sequence
  Synthetic Sequence
QVQFQESGPGLVKPSQTLSLTCTVSGYSITSDFAWNWIRQPPGNKLEWIGYISYSGDTNYNPSLKSRVTISRDTSKNQFSLKLSSVTAADTAVYFCAIRGPYSFTYWGQGTLVTVSS

SEQ ID NO: 30 - protein, 117 aa, Artificial Sequence
  Synthetic Sequence
QVQFQESGPGLVKPSQTLSLTCTVSGYSITSDFAWNWIRQFPGKGLEWIGYISYSGDTNYNPSLKSRVTISRDTSKNQFSLKLSSVTAADTAVYFCAIRGPYSFTYWGQGTLVTVSS

SEQ ID NO: 31 - protein, 117 aa, Artificial Sequence
  Synthetic Sequence
QVQFQESGPGLVKPSQTLSLTCTVSGYSITSDFAWNWIRQFPGNKLEWIGYISYSGDTNYNPSLKSRVTISRDTSKNQFSLKLSSVTAADTAVYFCAIRGPYSFTYWGQGTLVTVSS

SEQ ID NO: 32 - protein, 117 aa, Artificial Sequence
  Synthetic Sequence
DVQFQESGPGLVKPSQTLSLTCTVTGYSITSDFAWNWIRQPPGKGLEWIGYISYSGDTNYNPSLKSRVTITRDTSKNQFSLKLSSVTAADTATYFCAIRGPYSFTYWGQGTLVTVSS

SEQ ID NO: 33 - protein, 116 aa, Artificial Sequence
  Synthetic Sequence
QVQLQQSGPELVKPGALVKISCKASGFTFTSYDINWVRQRPGQGLEWIGWIYPGDGSTKYNEKFKGKATLTADKSSSTAYMQLSSLTSENSAVYFCTRSFYTMDYWGQGTSVTVSS

SEQ ID NO: 34 - protein, 120 aa, Artificial Sequence
  Synthetic Sequence
QVQLQQSGPELVRPGASVRMSCKASDYTFTNYWMHWVKQRPGQGLEWIGMFDPSNSVTRLNQKFKDKATLNVDKSSNTAYIQFSSLTFEDSAVYYCARTTSMIIGGFAYWGQGTLVTVSS

SEQ ID NO: 35 - protein, 117 aa, Artificial Sequence
  Synthetic Sequence
DVQFQESGPGLVKPSQSLSLTCTVTGYSITSDFAWNWIRQFPGNKLEWMGYISYSGDTNYNPSLKSRISITRDTSKNQFFLQLNSVTTEDTATYFCAIRGPYSFTYWGPGTLVTVSS

SEQ ID NO: 36 - protein, 112 aa, Artificial Sequence
  Synthetic Sequence
  MISC_FEATURE (34)..(34): Xaa1 is glycine (Gly) or alanine (Ala)
  MISC_FEATURE (41)..(41): Xaa2 is phenylalanine (Phe) or tyrosine (Tyr)
  MISC_FEATURE (54)..(54): Xaa3 is tyrosine (Tyr) or serine (Ser)
DIVMTQSPLSLPVTPGEPASISCRSSKSLLHSNXNTYLYWXLQKPGQSPQLLIXRMSNLASGVPDRFSGSGSGTDFTLKISRVEAEDVGVYYCMQHLEYPFTFGQGTKLEIK

SEQ ID NO: 37 - protein, 112 aa, Artificial Sequence
  Synthetic Sequence
DIVMTQSPLSLPVTPGEPASISCRSSKSLLHSNGNTYLYWYLQKPGQSPQLLIYRMSNLASGVPDRFSGSGSGTDFTLKISRVEAEDVGVYYCMQHLEYPFTFGQGTKLEIK

SEQ ID NO: 38 - protein, 112 aa, Artificial Sequence
  Synthetic Sequence
DIVMTQSPLSLPVTPGEPASISCRSSKSLLHSNGNTYLYWFLQKPGQSPQLLISRMSNLASGVPDRFSGSGSGTDFTLKISRVEAEDVGVYYCMQHLEYPFTFGQGTKLEIK

SEQ ID NO: 39 - protein, 112 aa, Artificial Sequence
  Synthetic Sequence
DIVMTQSPLSLPVTPGEPASISCRSSKSLLHSNANTYLYWFLQKPGQSPQLLISRMSNLASGVPDRFSGSGSGTDFTLKISRVEAEDVGVYYCMQHLEYPFTFGQGTKLEIK

SEQ ID NO: 40 - protein, 112 aa, Artificial Sequence
  Synthetic Sequence
  MISC_FEATURE (32)..(32): Xaa1 is serine (Ser) or arginine (Arg)
  MISC_FEATURE (34)..(34): Xaa2 is glycine (Gly) or alanine (Ala)
  MISC_FEATURE (43)..(43): Xaa3 is glutamine (Gln) or histidine (His)
DIVMTQTPLSLSVTPGQPASISCRSSKSLLHXNXITYFYWYLXKPGQPPQLLIYQMSNLASGVPDRFSGSGSGTDFTLKISRVEAEDVGVYYCAQNLELPLTFGGGTKVEIK

SEQ ID NO: 41 - protein, 112 aa, Artificial Sequence
  Synthetic Sequence
DIVMTQTPLSLSVTPGQPASISCRSSKSLLHSNGITYFYWYLQKPGQPPQLLIYQMSNLASGVPDRFSGSGSGTDFTLKISRVEAEDVGVYYCAQNLELPLTFGGGTKVEIK

SEQ ID NO: 42 - protein, 112 aa, Artificial Sequence
  Synthetic Sequence
DIVMTQTPLSLSVTPGQPASISCRSSKSLLHSNAITYFYWYLQKPGQPPQLLIYQMSNLASGVPDRFSGSGSGTDFTLKISRVEAEDVGVYYCAQNLELPLTFGGGTKVEIK

SEQ ID NO: 43 - protein, 112 aa, Artificial Sequence
  Synthetic Sequence
DIVMTQTPLSLSVTPGQPASISCRSSKSLLHRNAITYFYWYLQKPGQPPQLLIYQMSNLASGVPDRFSGSGSGTDFTLKISRVEAEDVGVYYCAQNLELPLTFGGGTKVEIK

SEQ ID NO: 44 - protein, 112 aa, Artificial Sequence
  Synthetic Sequence
DIVMTQTPLSLSVTPGQPASISCRSSKSLLHRNAITYFYWYLHKPGQPPQLLIYQMSNLASGVPDRFSGSGSGTDFTLKISRVEAEDVGVYYCAQNLELPLTFGGGTKVEIK

SEQ ID NO: 45 - protein, 107 aa, Artificial Sequence
  Synthetic Sequence
  MISC_FEATURE (64)..(64): Xaa1 is serine (Ser) or proline (Pro)
  MISC_FEATURE (71)..(71): Xaa2 is phenylalanine (Phe) or tyrosine (Tyr)
DIQMTQSPSSLSASVGDRVTITCRASQDINNYLNWYQQKPGKAPKLLIYYTSRLHSGVPSRFSXSGSGTDXTFTISSLQPEDIATYYCQQGHTLPWTFGGGTKVEIK

SEQ ID NO: 46 - protein, 107 aa, Artificial Sequence
  Synthetic Sequence
DIQMTQSPSSLSASVGDRVTITCRASQDINNYLNWYQQKPGKAPKLLIYYTSRLHSGVPSRFSGSGSGTDFTFTISSLQPEDIATYYCQQGHTLPWTFGGGTKVEIK

SEQ ID NO: 47 - protein, 107 aa, Artificial Sequence
  Synthetic Sequence
DIQMTQSPSSLSASVGDRVTITCRASQDINNYLNWYQQKPGKAPKLLIYYTSRLHSGVPSRFSPSGSGTDYTFTISSLQPEDIATYYCQQGHTLPWTFGGGTKVEIK

SEQ ID NO: 48 - protein, 112 aa, Artificial Sequence
  Synthetic Sequence
DIVMTQAAPSVPVTPGESVSISCRSSKSLLHSNGNTYLYWFLQRPGQSPQLLISRMSNLASGVPDRFSGSGSGTAFTLRISRVEAEDVGVYYCMQHLEYPFTFGSGTKLEIK

SEQ ID NO: 49 - protein, 112 aa, Artificial Sequence
  Synthetic Sequence
DIVMTQAAFSNPVTLGTSASISCRSSKSLLHSNGITYFYWYLQRPGQSPQLLIYQMSNLASGVPDRFSGSGSGTDFTLRISRVEAEDVGIYYCAQNLELPLTFGAGTKLELK

SEQ ID NO: 50 - protein, 107 aa, Artificial Sequence
  Synthetic Sequence
DIQMTQTTSSLSASLGDRVTINCRASQDINNYLNWYQQKPDGTIRLLIYYTSRLHSGVPSRFSPNGSGTDYSLTINNLEQEDIATYFCQQGHTLPWTFGGGTKLEIK

SEQ ID NO: 51 - protein, 117 aa, Artificial Sequence
  Synthetic Sequence
DVQFQESGPGLVKPSQTLSLTCTVTGYSITSDFAWNWIRQPPGKGLEWIGYISYSGDTNYNPSLKSRVTITRDTSKNQFSLKLSSVTAADTAVYFCAIRGPYSFTYWGQGTLVTVSS

SEQ ID NO: 52 - protein, 118 aa, Artificial Sequence
  Synthetic Sequence
DVQFQESGPGLVKPSQTLSLTCTVTGYSITADFAWNWIRQPPGKGLEWIGYISYSGDTNYNPSLKSRVTITRDTSKNQFSLKLSSVTAADTAVYFCAIRGPYSFTYWGQGTLVTVSSA

SEQ ID NO: 53 - protein, 118 aa, Artificial Sequence
  Synthetic Sequence
DLQFQESGPGLVKPSQTLSLTCTVTGYSITADFAWNWIRQPPGKGLEWIGYISYSGDTNYNPSLKSRVTITRDTSKNQFSLKLSSVTAADTAVYFCAIRGPYSFTYWGQGTLVTVSSA

SEQ ID NO: 54 - protein, 118 aa, Artificial Sequence
  Synthetic Sequence
DLQFQESGPGLVKPSQTLSLTCTVSRYSITADFAWNWIRQPPGKGLEWIGYISYSGDTNYNPSLKSRVTITRDTSKNQFSLKLSSVTAADTAVYFCAIRGPYSFTYWGQGTLVTVSSA

SEQ ID NO: 55 - protein, 109 aa, Artificial Sequence
  Synthetic Sequence
DIQMTQSPSSLSASVGDRVTITCRASQWINNYLNWYQQKPGKAPKLLIYYTSMLHSGVPSRFSPSGSGNDYTFTISSLQPEDIATYYCQQGHTLPWTFGGGTKVEIKRT

SEQ ID NO: 56 - protein, 116 aa, Artificial Sequence
  Synthetic Sequence
  MISC_FEATURE (4)..(4): Xaa1 is leucine (Leu) or phenylalanine (Phe)
  MISC_FEATURE (5)..(5): Xaa2 is valine (Val), methionine (Met), or leucine (Leu)
  MISC_FEATURE (44)..(44): Xaa3 is arginine (Arg) or glycine (Gly)
  MISC_FEATURE (56)..(56): Xaa4 is glycine (Gly), serine (Ser), or alanine (Ala)
  MISC_FEATURE (72)..(72): Xaa5 is arginine (Arg) or alanine (Ala)
  MISC_FEATURE (74)..(74): Xaa6 is threonine (Thr) or lysine (Lys)
  MISC_FEATURE (77)..(77): Xaa7 is serine (Ser) or asparagine (Asn)
  MISC_FEATURE (84)..(84): Xaa8 is serine (Ser) or alanine (Ala)
  MISC_FEATURE (95)..(95): Xaa9 is tyrosine (Tyr) or phenylalanine (Phe)
QVQXXQSGAEVKKPGASVKVSCKASGFTFTSYDINWVRQAPGQXLEWMGWIYPGDXSTKYNEKFKGRVTITXDXSAXTAYMELXSLRSEDTAVYXCTRSFYTMDYWGQGTTVTVSS

SEQ ID NO: 57 - protein, 118 aa, Artificial Sequence
  Synthetic Sequence
  MISC_FEATURE (1)..(1): Xaa1 is glutamine (Gln) or aspartic acid (Asp)
  MISC_FEATURE (2)..(2): Xaa2 is valine (Val) or leucine (Leu)
  MISC_FEATURE (4)..(4): Xaa3 is leucine (Leu) or phenylalanine (Phe)
  MISC_FEATURE (25)..(25): Xaa4 is threonine (Thr) or serine (Ser)
  MISC_FEATURE (26)..(26): Xaa5 is glycine (Gly) or arginine (Arg)
  MISC_FEATURE (31)..(31): Xaa6 serine (Ser) or alanine (Ala)
  MISC_FEATURE (41)..(41): Xaa7 is proline (Pro) or phenylalanine (Phe)
  MISC_FEATURE (44)..(44): Xaa8 is lysine (Lys) or asparagine (Asn)
  MISC_FEATURE (45)..(45): Xaa9 is glycine (Gly) or lysine (Lys)
  MISC_FEATURE (71)..(71): Xaa10 is serine (Ser) or threonine (Thr)
  MISC_FEATURE (72)..(72): Xaa11 is valine (Val) or arginine (Arg)
  MISC_FEATURE (93)..(93): Xaa12 is threonine (Thr) or valine (Val)
  MISC_FEATURE (95)..(95): Xaa13 is tyrosine (Tyr) or phenylalanine (Phe)
  MISC_FEATURE (118)..(118): Xaa14 is is alanine (Ala) or absent
XXQXQESGPGLVKPSQTLSLTCTVXXYSITXDFAWNWIRQXPGXXLEWIGYISYSGDTNYNPSLKSRVTIXXDTSKNQFSLKLSSVTAADTAXYXCAIRGPYSFTYWGQGTLVTVSSX

SEQ ID NO: 58 - protein, 109 aa, Artificial Sequence
  Synthetic Sequence
  MISC_FEATURE (28)..(28): Xaa1 is aspartic acid (Asp) or tryptophan (Trp)
  MISC_FEATURE (53)..(53): Xaa2 is arginine (Arg) or methionine (Met)
  MISC_FEATURE (64)..(64): Xaa3 is glycine (Gly), serine (Ser) or proline (Pro)
  MISC_FEATURE (69)..(69): Xaa4 is threonine (Thr) or asparagine (Asn)
  MISC_FEATURE (71)..(71): Xaa5 is phenylalanine (Phe) or tyrosine (Tyr)
  MISC_FEATURE (108)..(108): Xaa6 is arginine (Arg) or absent
  MISC_FEATURE (109)..(109): Xaa7 is threonine (Thr) or absent
DIQMTQSPSSLSASVGDRVTITCRASQXINNYLNWYQQKPGKAPKLLIYYTSXLHSGVPSRFSXSGSGXDXTFTISSLQPEDIATYYCQQGHTLPWTFGGGTKVEIKXX

SEQ ID NO: 59 - protein, 9 aa, Artificial Sequence
  synthetic
FTFTSYDIN

SEQ ID NO: 60 - protein, 17 aa, Artificial Sequence
  synthetic
WIYPGDGSTKYNEKFKG

SEQ ID NO: 61 - protein, 17 aa, Artificial Sequence
  synthetic
WIYPGDSSTKYNEKFKG

SEQ ID NO: 62 - protein, 17 aa, Artificial Sequence
  synthetic
WIYPGDASTKYNEKFKG

SEQ ID NO: 63 - protein, 7 aa, Artificial Sequence
  synthetic
SFYTMDY

SEQ ID NO: 64 - protein, 9 aa, Artificial Sequence
  synthetic
YTFTNYWMH

SEQ ID NO: 65 - protein, 9 aa, Artificial Sequence
  synthetic
YTFTNYWMN

SEQ ID NO: 66 - protein, 9 aa, Artificial Sequence
  synthetic
YTFTNYWMY

SEQ ID NO: 67 - protein, 9 aa, Artificial Sequence
  synthetic
YTFTNYYMN

SEQ ID NO: 68 - protein, 17 aa, Artificial Sequence
  synthetic
MFDPSNSVTRLNQKFKD

SEQ ID NO: 69 - protein, 17 aa, Artificial Sequence
  synthetic
MFEPSNAVTRLNQKFKD

SEQ ID NO: 70 - protein, 17 aa, Artificial Sequence
  synthetic
MFHPSNAVTRLNQKFKD

SEQ ID NO: 71 - protein, 17 aa, Artificial Sequence
  synthetic
MFHPTGDVTRLNQKFKD

SEQ ID NO: 72 - protein, 11 aa, Artificial Sequence
  synthetic
TTSMIIGGFAY

SEQ ID NO: 73 - protein, 10 aa, Artificial Sequence
  synthetic
YSITSDFAWN

SEQ ID NO: 74 - protein, 10 aa, Artificial Sequence
  synthetic
YSITADFAWN

SEQ ID NO: 75 - protein, 16 aa, Artificial Sequence
  synthetic
YISYSGDTNYNPSLKS

SEQ ID NO: 76 - protein, 8 aa, Artificial Sequence
  synthetic
RGPYSFTY

SEQ ID NO: 77 - protein, 16 aa, Artificial Sequence
  synthetic
RSSKSLLHSNGNTYLY

SEQ ID NO: 78 - protein, 16 aa, Artificial Sequence
  synthetic
RSSKSLLHSNANTYLY

SEQ ID NO: 79 - protein, 6 aa, Artificial Sequence
  synthetic
RMSNLA

SEQ ID NO: 80 - protein, 9 aa, Artificial Sequence
  synthetic
MQHLEYPFT

SEQ ID NO: 81 - protein, 16 aa, Artificial Sequence
  synthetic
RSSKSLLHSNGITYFY

SEQ ID NO: 82 - protein, 16 aa, Artificial Sequence
  synthetic
RSSKSLLHSNAITYFY

SEQ ID NO: 83 - protein, 16 aa, Artificial Sequence
  synthetic
RSSKSLLHRNAITYFY

SEQ ID NO: 84 - protein, 7 aa, Artificial Sequence
  synthetic
QMSNLAS

SEQ ID NO: 85 - protein, 9 aa, Artificial Sequence
  synthetic
AQNLELPLT

SEQ ID NO: 86 - protein, 11 aa, Artificial Sequence
  synthetic
RASQDINNYLN

SEQ ID NO: 87 - protein, 11 aa, Artificial Sequence
  synthetic
RASQWINNYLN

SEQ ID NO: 88 - protein, 7 aa, Artificial Sequence
  synthetic
YTSRLHS

SEQ ID NO: 89 - protein, 7 aa, Artificial Sequence
  synthetic
YTSMLHS

SEQ ID NO: 90 - protein, 9 aa, Artificial Sequence
  synthetic
QQGHTLPWT

(19) United States
(12) Patent Application Publication
(10) Pub. No.: US 2024/0216506 A1
(43) Pub. Date: Jul. 4, 2024

(54) ANTIBODY COMPOSITION

(71) Applicant: AnaptysBio, Inc., San Diego, CA (US)

(72) Inventors: Alex Praseuth, San Diego, CA (US); Kevin Zen, Del Mar, CA (US); Laurence Altobell, III, San Diego, CA (US); Margaret H. Marino, San Diego, CA (US)

(21) Appl. No.: 18/560,317

(22) PCT Filed: May 12, 2022
(86) PCT No.: PCT/US2022/029043

§ 371 Date: Nov. 10, 2023

Related U.S. Application Data
(60) Provisional application No. 63/187,476, filed on May 12, 2021.

(51) Int. Cl. A61K 39/395; A61K 9/08; A61K 47/22; A61K 47/26; C07K 16/24; C07K 16/28

(52) U.S. Cl. CPC A61K 39/39591; A61K 9/08; A61K 47/22; A61K 47/26; C07K 16/244; C07K 16/2866

(57) ABSTRACT

The invention relates to a stable liquid aqueous pharmaceutical formulation comprising an anti-interleukin 36 receptor (IL-36R) antibody or antigen binding antibody fragment; a buffer; a stabilizer; and a surfactant, and methods of treating a subject with the pharmaceutical formulation.

### CROSS-REFERENCE TO A RELATED APPLICATION

This patent application claims the benefit of U.S. Provisional Patent Application No. 63/187,476, filed May 12, 2021, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 76,894 Byte ASCII (Text) file named “762991_ST25.TXT,” created on May 12, 2022.

### BACKGROUND OF THE INVENTION

Formulations or compositions containing molecules such as antibodies require specified formulations to make them suitable to patient administration as well as stability during transportation, storage, and use. Antibody formulations can be negatively affected by denaturation, oxidation, and aggregation, especially when a high concentration of antibody is desired, which often occurs when the antibody formulation is used for a pharmaceutical or therapeutic purpose. There is a need to provide a stable liquid aqueous pharmaceutical formulation for antibodies, and particularly interleukin-36 (IL-36) antibodies to help meet the medical need of patients suffered by diseases mediated by IL-36 and its receptor.

### BRIEF SUMMARY OF THE INVENTION

In one embodiment, the invention provides a stable liquid pharmaceutical formulation comprising an antibody or antigen binding antibody fragment; a buffer; a stabilizer comprising proline and/or sorbitol, and a non-ionic surfactant. Related compositions and methods for the use thereof also are provided.

### BRIEF DESCRIPTION OF THE SEVERAL VIEWS OF THE DRAWING(S)

FIG. 1A shows images of the visual appearance of 1 mL sample F01 at 0, 5, 6, 7, and 8 days as compared to water (leftmost vial) when stored at 45° C.

FIG. 1B shows images of the visual appearance of 1 mL sample F02A at 0, 5, 6, 7, and 8 days as compared to water (leftmost vial) when stored at 45° C.

FIG. 1C shows images of the visual appearance of 1 mL sample F02B at 0, 5, 6, 7, and 8 days as compared to water (leftmost vial) when stored at 45° C.

FIG. 1D shows images of the visual appearance of 1 mL sample F02C at 0, 5, 6, 6, and 8 days as compared to water (leftmost vial) when stored at 45° C.

FIG. 2 is a plot of turbidity by A350 (AU) vs. time (day) of IL-36R formulations F01, F02A, F02B, and F02C stored at 45° C. The lines represent linear models of each formulation.

FIG. 3 is a plot of purity by SEC-HPLC (% main) vs. time (day) of IL-36R formulations, F01, F02A, F02B, and F02C stored at 45° C. The lines represent linear models of each formulation.

### DETAILED DESCRIPTION OF THE INVENTION

Provided herein is a stable liquid aqueous pharmaceutical formulation or composition comprising water; an antibody or antigen binding antibody fragment; a buffer; a stabilizer comprising proline and/or sorbitol, and a non-ionic surfactant. The terms “formulation” and “composition” (e.g., “pharmaceutical formulation” or “pharmaceutical composition”) as used herein have the same meaning are used interchangeably.

### Buffer and pH

Any suitable buffer can be used; however, in some embodiments, the buffer is a histidine buffer. The buffer can be used at a suitable concentration to maintain the desired pH, generally from about 5.0 to about 6.5 (e.g., about 5.5-6.2 or about 5.8-6.0). In some embodiments, the formulation may have a pH of about 5.0, about 5.1 about 5.2, about 5.3, about 5.4, about 5.5 about 5.6 about 5.7 about 5.8, about 5.9, about 6.0, about 6.1 about 6.2, about 6.3, about 6.4, or about 6.5. In some embodiments the pH is 6.0±0.4, 6.0±0.3, 6.0±0.2, 6.0±0.1, about 6.0 or 6.0.

In some embodiments, the buffer is a histidine buffer, and the histidine is present in the formulation at a concentration of about 5-35 mM (e.g., about 5 mM, about 10 mM, about 15 mM, about 20 mM, about 25 mM, about 30 mM, about 35 mM, or a range defined by any two of the foregoing values), or about 5-20 mM (e.g., about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, or a range defined by any two of the foregoing values). In some embodiments, the pharmaceutical formulation comprises about 7-25 mM histidine, such as about 7-15 mM histidine; or about 10-25 mM histidine, such as about 10-15 mM histidine. In some embodiments, the pharmaceutical formulation comprises about 9-11 mM histidine (e.g., about 10 mM histidine) or about 24-26 mM histidine (e.g., about 25 mM histidine).

### Stabilizer

In another embodiment, the pharmaceutical formulation further comprises a stabilizer comprising, consisting essentially of, or consisting of proline and/or sorbitol. The stabilizer can have any suitable concentration of proline and/or sorbitol. In some embodiments, the stabilizer comprises, consists essentially of, or consists of proline. In some embodiments, the proline is present in the formulation at a concentration of about 100 to 300 mM (e.g., about 100 mM, about 125 mM, about 150 mM, about 175 mM, about 200 mM, about 225 mM, about 250 mM, about 275 mM, about 300 mM, or a range defined by any two of the foregoing values), such as from about 270 mM to about 300 mM (e.g., about 270 mM, about 275 mM, about 280 mM, about 285 mM, about 290 mM, about 295 mM, about 300 mM, or a range defined by any two of the foregoing values), or about 275-285 mM. In other embodiments, the stabilizer comprises, consists essentially of, or consists of sorbitol. In some embodiments, the sorbitol is present in the formulation at a concentration of about 100 to 300 mM (e.g., about 100 mM, about 125 mM, about 150 mM, about 175 mM, about 200 mM, about 225 mM, about 250 mM, about 275 mM, about 300 mM, or a range defined by any two of the foregoing values), such as from about 270 mM to about 300 mM (e.g., about 270 mM, about 275 mM, about 280 mM, about 285 mM, about 290 mM, about 295 mM, about 300 mM, or a range defined by any two of the foregoing values). In still other embodiment, the stabilizer comprises, consists essentially of, or consists of proline and sorbitol. In some embodiments, the combined concentration of the sorbitol and proline in the formulation is about 100 to 300 mM (e.g., about 100 mM, about 125 mM, about 150 mM, about 175 mM, about 200 mM, about 225 mM, about 250 mM, about 275 mM, about 300 mM, or a range defined by any two of the foregoing values), such as from about 270 mM to about 300 mM (e.g., about 270 mM, about 275 mM, about 280 mM, about 285 mM, about 290 mM, about 295 mM, about 300 mM, or a range defined by any two of the foregoing values). For example, the formulation can comprise about 50-80 mM sorbitol and about 180-250 mM proline (e.g., about 65-75 mM sorbitol, or about 70 mm sorbitol) and about 205-215 mM proline (e.g., about 210 mM proline).

### Surfactant

The stable liquid aqueous pharmaceutical formulations also comprise a non-ionic surfactant. In one embodiment, the surfactant is a polysorbate. In a preferred embodiment, the surfactant is selected from the group consisting of polysorbate 20, polysorbate 21, polysorbate 40, polysorbate 60, polysorbate 61, polysorbate 65, polysorbate 80, polysorbate 81, polysorbate 85, and mixtures thereof, and is preferably selected from polysorbate 20 or polysorbate 80, or mixtures thereof. In a more preferred embodiment, the surfactant is polysorbate 20.

In certain embodiments the surfactant is present in a concentration of about 0.01% to about 0.1% (e.g., about 0.01%, about 0.02%, about 0.03%, about 0.04%, about 0.05%, about 0.06%, about 0.07%, about 0.08%, about 0.09%, about 0.1%, or a range defined by any two of the foregoing values), such as about 0.01% to about 0.05% or about 0.03% to about 0.07% (e.g., about 0.01%, about 0.015%, about 0.02%, about 0.025%, about 0.03%, about 0.035%, about 0.04%, about 0.045%, about 0.05%, about 0.07% or a range defined by any two of the foregoing values).

### Viscosity

The formulation can have any viscosity suitable for the intended use (e.g., parenteral injection, such as subcutaneous injection). In one embodiment, the viscosity of the pharmaceutical formulation is less than or equal to about 15 cPoise (“cps” or “cP”). In another embodiment the viscosity of the pharmaceutical formulation is between about 1 cP and about 18 cP (e.g., about 1 cP, about 2 cP, about 3 cP, about 4 cP, about 5 cP, about 6 cP, about 7 cP, about 8 cP, about 9 cP, about 10 cP, about 11 cP, about 12 cP, about 13 cP, about 14 cP, about 15 cP, about 16 cP, about 17 cP, about 18 cP, or a range defined by any two of the foregoing values).

### Osmolality

The formulation can have any suitable osmolality. In one embodiment, the osmolality of the pharmaceutical formulation is between about 250 mOsm/kg and 450 mOsm/kg (e.g., about 250 mOsm/kg, about 275 mOsm/kg, about 300 mOsm/kg, about 325 mOsm/kg, about 350 mOsm/kg, about 375 mOsm/kg, about 400 mOsm/kg, about 425 mOsm/kg, about 450 mOsm/kg, or a range defined by any two of the foregoing values). In a particular embodiment, the osmolality of the pharmaceutical formulation is between about 300 mOsm/kg and 400 mOsm/kg (e.g., about 300 mOsm/kg, about 310 mOsm/kg, about 320 mOsm/kg, about 330 mOsm/g, about 340 mOsm/kg, about 350 mOsm/kg, about 360 mOsm/kg, about 370 mOsm/kg, about 380 mOsm/kg, about 390 mOsm/kg, about 400 mOsm/kg, or a range defined by any two of the foregoing values).

In some embodiments, the formulation comprises less than 10 mM (e.g., less than 5 mM, less than 1 mM, or less than 0.5 mM) of sodium chloride (NaCl), or is substantially or completely free of NaCl. In some embodiments, the formulation comprises less than 10 mM (e.g., less than 5 mM, less than 1 mM, or less than 0.5 mM) of any tonicity agents, or is substantially or completely free of any tonicity agents. In some embodiments, the formulation consists essentially of or consists of the antibody or antigen binding antibody fragment, buffer, stabilizer, non-ionic surfactant, and water, as described herein.

### Antibody or Antibody-Fragment

The stable liquid aqueous pharmaceutical formulations of the present invention may comprise any antibody or antigen binding antibody fragment. In some embodiments, the formulation comprises an anti-IL-36R antibody or antigen-binding antibody fragment, particularly an antibody as described in detail herein.

In an embodiment, the IL-36R antibody is an antibody or antigen-binding antibody fragment. An antibody or antigen-binding antibody fragment comprises, consist of, or consists essentially of an immunoglobulin heavy chain polypeptide and an immunoglobulin light chain polypeptide, or at least the variable regions (e.g., antigen-binding fragments) thereof.

A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (CH1, CH2, and CH3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, NY (2001)).

The framework regions are connected by three complementarity determining regions (CDRs). As discussed above, the three CDRs, known as CDR1, CDR2, and CDR3, form the “hypervariable region” of an antibody, which is responsible for antigen binding. The CDR regions also can be referred to using an “H” or “L” in the nomenclature to denote the heavy or light chain, respectively, i.e., CDRH1, CDRH2, CDRH3, CDRL1, CDRL2, or CDRL3. The CDRs of a given Ig sequence can be determined by any of several conventional numbering schemes, such as Kabat, Chothia, Martin (Enhanced Chothia), IGMT, or AHo (see, e.g., Kabat, et al., Sequences of Proteins of Immunological Interest, U.S. Department of Health and Human Services, NIH (1991); Chothia, et al., Canonical Structures for the Hypervariable Regions of Immunoglobulins, J. Mol. Biol., 196:901-917 (1987); Al-Lazikani et al., Standard Conformations for the Canonical Structures of Immunoglobulins, J. Mol. Biol., 273:927-948 (1997): Abhinandan et al., Analysis and Improvements to Kabat and Structurally Correct Numbering of Antibody Variable Domains, Mol. Immunol., 45: 3832-3839 (2008); Lefranc et al., The IMGT unique numbering for immunoglobulins, T cell Receptors and Ig-like domains, The Immunologist, 7: 132-136 (1999); Lefranc et al., IMGT unique numbering for immunoglobulin and T cell receptor variable domains and I superfamily V-like domains, Dev. Comp. Immunol., 27: 55-77 (2003); and Honegger et al., Yet another numbering scheme for immunoglobulin variable domains: an automatic modeling and analysis tool, J. Mol. Biol. 309: 657-670 (2001).

In one embodiment, the immunoglobulin heavy chain variable region comprises, consists of, or consists essentially of the amino acid sequence of Gln Val Gln Xaa1 Xaa2 Gln Ser Gly Ala Glu Val Lys Pro Gly Ala Ser Val Lys Val Ser Cys Lys Ala Ser Gly Phe Thr Phe Thr Ser Tyr Asp Ile Asn Trp Val Arg Gln Ala Pro Gly Gln Xaa3 Leu Glu Trp Met Gly Trp Ile Tyr Pro Gly Asp Xaa4 Ser Thr Lys Tyr Asn Glu Lys Phe Lys Gly Arg Val Thr Ile Thr Xaa5 Asp Xaa6 Ser Ala Xaa7 Thr Ala Tyr Met Glu Leu Xaa8 Ser Leu Arg Ser Glu Asp Thr Ala Val Tyr Xaa9 Cys Thr Arg Ser Phe Tyr Thr Met Asp Tyr Trp Gly Gln Gly Thr Val Thr Val Ser (SEQ ID NO: 56), or at least the CDRs thereof, wherein (a) Xaa1 is leucine (Leu) or phenylalanine (Phe), (b) Xaa2 is valine (Val), methionine (Met), or leucine (Leu), (c) Xaa3 is arginine (Arg) or glycine (Gly), (d) Xaa4 is glycine (Gly), serine (Ser), or alanine (Ala), (e) Xaa5 is arginine (Arg) or alanine (Ala), (f) Xaa6 is threonine (Thr) or lysine (Lys), (g) Xaa7 is serine (Ser) or asparagine (Asn), (h) Xaa8 is serine (Ser) or alanine (Ala), and (i) Xaa9 is tyrosine (Tyr) or phenylalanine (Phe). In some embodiments, the immunoglobulin heavy chain polypeptide comprises, consists of, or consists essentially of the amino acid sequence Gln Val Gln Xaa1 Xaa2 Gln Ser Gly Ala Glu Val Lys Lys Pro Gly Ala Ser Val Lys Val Ser Cys Lys Ala Ser Gly Phe Thr Phe Thr Ser Tyr Asp Ile Asn Trp Val Arg Gln Ala Pro Gly Gln Xaa3 Leu Glu Trp Met Gly Trp Ile Tyr Pro Gly Asp Xaa4 Ser Thr Lys Tyr Asn Glu Lys Phe Lys Gly Arg Val Thr Ile Thr Xaa5 Asp Xaa6 Ser Ala Ser Thr Ala Tyr Met Glu Leu Xaa7 Ser Leu Arg Ser Glu Asp Thr Ala Val Tyr Xaa8 Cys Thr Arg Ser Phe Tyr Thr Met Asp Tyr Trp Gly Gln Gly Thr Thr Val Thr Val Ser Ser (SEQ ID NO: 1), or at least the CDRs thereof, wherein (a) Xaa1 is leucine (Leu) or phenylalanine (Phe), (b) Xaa2 is valine (Val), methionine (Met), or leucine (Leu), (c) Xaa3 is arginine (Arg) or glycine (Gly), (d) Xaa4 is glycine (Gly), serine (Ser), or alanine (Ala), (e) Xaa5 is arginine (Arg) or alanine (Ala), (f) Xaa6 is threonine (Thr) or lysine (Lys), (g) Xaa7 is serine (Ser) or alanine (Ala), and (h) Xaa8 is tyrosine (Tyr) or phenylalanine (Phe).

The heavy chain polypeptide can comprise, consist of, or consist essentially of the amino acid sequence of SEQ ID NO: 56 or SEQ ID NO: 1, or at least the CDRs thereof, with any one of the aforementioned amino acid substitutions in any suitable combination. In one embodiment, the immunoglobulin heavy chain polypeptide comprises, consists of, or consists essentially of an amino acid sequence of any one of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or SEQ ID NO: 14, or at least the CDRs thereof.

In some embodiments, the IL-36R antibody or antibody fragment comprises: a CDR1 of the heavy chain variable region (HCDR1) comprising, consisting of, or consisting essentially of the amino acid sequence Phe Thr Phe Thr Ser Tyr Asp Ile Asn (SEQ ID NO: 59); a CDR2 of the heavy chain variable region (HCDR2) comprising, consisting of, or consisting essentially of the amino acid sequence of (a) Trp Ile Tyr Pro Gly Asp Gly Ser Thr Lys Tyr Asn Glu Lys Phe Lys Gly (SEQ ID NO: 60); (b) Trp Ile Tyr Pro Gly Asp Ser Ser Thr Lys Tyr Asn Glu Lys Phe Lys Gly (SEQ ID NO: 61); or (c) Trp Ile Tyr Pro Gly Asp Ala Ser Thr Lys Tyr Asn Glu Lys Phe Lys Gly (SEQ ID NO: 62); and/or a CDR3 of the heavy chain variable region (HCDR3) comprising, consisting of, or consisting essentially of the amino acid sequence Ser Phe Tyr Thr Met Asp Tyr (SEQ ID NO: 63).

In another embodiment, the immunoglobulin heavy chain variable region comprises, consists of, or consists essentially of the amino acid sequence Gln Val Gln Leu Val Gln Ser Gly Ala Glu Val Lys Lys Pro Gly Ala Ser Val Lys Val Ser Cys Lys Ala Ser Gly Tyr Thr Phe Thr Asn Tyr Xaa1 Met Xaa2 Trp Val Arg Gln Ala Pro Xaa3 Gln Gly Leu Glu Trp Met Gly Met Phe Xaa4 Pro Xaa5 Xaa6 Xaa7 Val Thr Arg Leu Asn Gln Lys Phe Lys Asp Arg Val Thr Met Thr Arg Asp Thr Ser Thr Ser Thr Val Tyr Met Glu Leu Ser Ser Leu Arg Ser Glu Asp Thr Ala Val Tyr Tyr Cys Ala Arg Thr Thr Ser Met Ile Ile Gly Gly Phe Ala Tyr Trp Gly Gln Gly Thr Leu Val Thr Val Ser Ser (SEQ ID NO: 15), or at least the CDRs thereof, wherein (a) Xaa1 is tryptophan (Trp) or tyrosine (Tyr), (b) Xaa2 is histidine (His), asparagine (Asn), or tyrosine (Tyr), (c) Xaa3 is glycine (Gly) or arginine (Arg), (d) Xaa4 is aspartic acid (Asp), glutamic acid (Glu), or histidine (His), (e) Xaa5 is serine (Ser), threonine (Thr), or tyrosine (Tyr), (f) Xaa6 is asparagine (Asn) or glycine (Gly), and (g) Xaa7 is serine (Ser), alanine (Ala), or aspartic acid (Asp).

The heavy chain variable region can comprise, consist of, or consist essentially of the amino acid sequence of SEQ ID NO: 15, or at least the CDRs thereof, with one of the aforementioned amino acid substitutions in any suitable combination. In one embodiment, the immunoglobulin heavy chain polypeptide comprises, consists of, or consists essentially of an amino acid sequence of any one of SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, or SEQ ID NO: 24, or at least the CDRs thereof.

In one embodiment, the IL-36 antibody or antibody fragment comprises a heavy chain variable region comprising: an HCDR1 comprising, consisting of, or consisting essentially of the amino acid sequence selected from the group consisting of (a) Tyr Thr Phe Thr Asn Tyr Trp Met His (SEQ ID NO: 64); (b) Tyr Thr Phe Thr Asn Tyr Trp Met Asn (SEQ ID NO: 65); (c) Tyr Thr Phe Thr Asn Tyr Trp Met Tyr (SEQ ID NO: 66); and (d) Tyr Thr Phe Thr Asn Tyr Tyr Met Asn (SEQ ID NO: 67); an HCDR2 comprising, consisting of, or consisting essentially of the amino acid sequence selected from the group consisting of (a) Met Phe Asp Pro Ser Asn Ser Val Thr Arg Leu Asn Gln Lys Phe Lys Asp (SEQ ID NO: 68); (b) Met Phe Glu Pro Ser Asn Ala Val Thr Arg Leu Asn Gln Lys Phe Lys Asp (SEQ ID NO: 69); (c) Met Phe His Pro Ser Asn Ala Val Thr Arg Leu Asn Gln Lys Phe Lys Asp (SEQ ID NO: 70); and (d) Met Phe His Pro Thr Gly Asp Val Thr Arg Leu Asn Gln Lys Phe Lys Asp (SEQ ID NO: 71); and/or an HCDR3 comprising, consisting of, or consisting essentially of the amino acid sequence Thr Thr Ser Met Ile Ile Gly Gly Phe Ala Tyr (SEQ ID NO: 72).

In a further embodiment, the immunoglobulin heavy chain variable region comprises, consists of, or consists essentially of the amino acid sequence of Xaa1 Xaa2 Gln Xaa3 Gln Glu Ser Gly Pro Gly Leu Val Lys Pro Ser Gln Thr Leu Ser Leu Thr Cys Thr Val Xaa4 Xaa5 Tyr Ser Ile Thr Xaa6 Asp Phe Ala Trp Asn Trp Ile Arg Gln Xaa7 Pro Gly Xaa8 Xaa9 Leu Glu Trp Ile Gly Tyr Ile Ser Tyr Ser Gly Asp Thr Asn Tyr Asn Pro Ser Leu Lys Ser Arg Val Thr Ile Xaa10 Xaa11 Asp Thr Ser Lys Asn Gln Phe Ser Leu Lys Leu Ser Ser Val Thr Ala Ala Asp Thr Ala Xaa12 Tyr Xaa13 Cys Ala Ile Arg Gly Pro Tyr Ser Phe Thr Tyr Trp Gly Gln Gly Thr Leu Val Thr Val Ser Ser Xaa14 (SEQ ID NO: 57), or at least the CDRs thereof, wherein Xaa1 is glutamine (Gln) or aspartic acid (Asp); Xaa2 is valine (Val) or leucine (Leu); Xaa3 is leucine (Leu) or phenylalanine (Phe); Xaa4 is threonine (Thr) or serine (Ser); Xaa5 is glycine (Gly) or arginine (Arg); Xaa6 serine (Ser) or alanine (Ala); Xaa7 is proline (Pro) or phenylalanine (Phe); Xaa8 is lysine (Lys) or asparagine (Asn); Xaa9 is glycine (Gly) or lysine (Lys); Xaa10 is serine (Ser) or threonine (Thr); Xaa11 is valine (Val) or arginine (Arg); Xaa12 is threonine (Thr) or valine (Val); Xaa13 is tyrosine (Tyr) or phenylalanine (Phe); and Xaa14 is alanine (Ala) or absent. In some embodiments, the heavy chain variable region comprises, consists of, or consists essentially of the amino acid sequence Xaa1 Val Gln Xaa2 Gln Glu Ser Gly Pro Gly Leu Val Lys Pro Ser Gln Thr Leu Ser Leu Thr Cys Thr Val Xaa3 Gly Tyr Ser Ile Thr Ser Asp Phe Ala Trp Asn Trp Ile Arg Gln Xaa4 Pro Gly Xaa5 Xaa6 Leu Glu Trp Ile Gly Tyr Ile Ser Tyr Ser Gly Asp Thr Asn Tyr Asn Pro Ser Leu Lys Ser Arg Val Thr Ile Xaa7 Xaa8 Asp Thr Ser Lys Asn Gln Phe Ser Leu Lys Leu Ser Ser Val Thr Ala Ala Asp Thr Ala Val Tyr Xaa9 Cys Ala Ile Arg Gly Pro Tyr Ser Phe Thr Tyr Trp Gly Gln Gly Thr Leu Val Thr Val Ser Ser (SEQ ID NO: 25), or at least the CDRs thereof, wherein (a) Xaa1 is glutamine (Gln) or aspartic acid (Asp), (b) Xaa2 is leucine (Leu) or phenylalanine (Phe), (c) Xaa3 is threonine (Thr) or serine (Ser), (d) Xaa4 is proline (Pro) or phenylalanine (Phe), (e) Xaa5 is lysine (Lys) or asparagine (Asn), (f) Xaa6 is glycine (Gly) or lysine (Lys), (g) Xaa7 is serine (Ser) or threonine (Thr), (h) Xaa8 is valine (Val) or arginine (Arg), and (i) Xaa9 is tyrosine (Tyr) or phenylalanine (Phe).

In some embodiments, the heavy chain variable region can comprise, consist of, or consist essentially of the amino acid sequence of SEQ ID NO: 57 or SEQ ID NO: 25, or at least the CDRs thereof, with one or more of the aforementioned amino acid substitutions in any suitable combination. In one embodiment, the immunoglobulin heavy chain variable region comprises, consists of, or consists essentially of an amino acid sequence of any one of SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 51, SEQ ID NO: 52, SEQ ID NO: 53, or SEQ ID NO: 54, or at least the CDRs thereof.

In additional embodiments, the IL-36 antibody or antibody fragment may comprise an HCDR1 comprising, consisting of, or consisting essentially of the amino acid sequence selected from the group consisting of (a) Tyr Ser Ile Thr Ser Asp Phe Ala Trp Asn (SEQ ID NO: 73); and (b) Tyr Ser Ile Thr Ala Asp Phe Ala Trp Asn (SEQ ID NO: 74); an HCDR2 comprising, consisting of, or consisting essentially of the amino acid sequence Tyr Ile Ser Tyr Ser Gly Asp Thr Asn Tyr Asn Pro Ser Leu Lys Ser (SEQ ID NO: 75); and/or an HCDR3 comprising, consisting of, or consisting essentially of the amino acid sequence Arg Gly Pro Tyr Ser Phe Thr Tyr (SEQ ID NO: 76).

In another embodiment, the IL-36 antibody or antibody fragment comprises an immunoglobulin heavy chain polypeptide which comprises, consists of, or consists essentially of the amino acid sequence of SEQ ID NO: 33, SEQ ID NO: 34, or SEQ ID NO: 35, or at least the CDRs thereof.

In some embodiments, the immunoglobulin heavy chain polypeptide comprises an amino acid sequence that is at least 90% identical (e.g., at least 91%, at least 92%, at least 93%, at least 94%, at least 95%, at least 96%, at least 97%, at least 98%, at least 99%, or 100% identical) to any of the foregoing variable region sequences. Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FASTM, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

In addition to a heavy chain variable region as described herein, the IL-36R antibody or antibody fragment comprises an immunoglobulin light chain variable region that comprises, consists of, or consists essentially of the amino acid sequence Asp Ile Val Met Thr Gln Ser Pro Leu Ser Leu Pro Val Thr Pro Gly Glu Pro Ala Ser Ile Ser Cys Arg Ser Ser Lys Ser Leu Leu His Ser Asn Xaa1 Asn Thr Tyr Leu Tyr Trp Xaa2 Leu Gln Lys Pro Gly Gln Ser Pro Gln Leu Leu Ile Xaa3 Arg Met Ser Asn Leu Ala Ser Gly Val Pro Asp Arg Phe Ser Gly Ser Gly Ser Gly Thr Asp Phe Thr Leu Lys Ile Ser Arg Val Glu Ala Glu Asp Val Gly Val Tyr Tyr Cys Met Gln His Leu Glu Tyr Pro Phe Thr Phe Gly Gln Gly Thr Lys Leu Glu Ile Lys (SEQ ID NO: 36), or at least the CDRs thereof, wherein (a) Xaa1 is glycine (Gly) or alanine (Ala), (b) Xaa2 is phenylalanine (Phe) or tyrosine (Tyr), and (c) Xaa3 is tyrosine (Tyr) or serine (Ser).

The light chain variable region can comprise, consist of, or consist essentially of the amino acid sequence of SEQ ID NO: 36, or at least the CDRs thereof, with one or more of the aforementioned amino acid substitutions in any suitable combination. In one embodiment, the isolated immunoglobulin light chain variable region comprises, consists of, or consists essentially of an amino acid sequence of any one of SEQ ID NO: 37, SEQ ID NO: 38, or SEQ ID NO: 39, or at least the CDRs thereof.

In some embodiments, the IL-36R binding agent comprises a CDR1 of the light chain variable region (LCDR1) comprising, consisting of, or consisting essentially of the amino acid sequence selected of (a) Arg Ser Ser Lys Ser Leu Leu His Ser Asn Gly Asn Thr Tyr Leu Tyr (SEQ ID NO: 77); or (b) Arg Ser Ser Lys Ser Leu Leu His Ser Asn Ala Asn Thr Tyr Leu Tyr (SEQ ID NO: 78); a CDR2 of the light chain variable region (LCDR2) comprising, consisting of, or consisting essentially of the amino acid sequence Arg Met Ser Asn Leu Ala Ser (SEQ ID NO: 79); and a CDR3 of the light chain variable region (LCDR3) comprising, consisting of, or consisting essentially of the amino acid sequence Met Gln His Leu Glu Tyr Pro Phe Thr (SEQ ID NO: 80).

In some embodiments, the immunoglobulin light chain variable region comprises, consists of, or consists essentially of the amino acid sequence Asp Ile Val Met Thr Gln Thr Pro Leu Ser Leu Ser Val Thr Pro Gly Gln Pro Ala Ser Ile Ser Cys Arg Ser Ser Lys Ser Leu Leu His Xaa1 Asn Xaa2 Ile Thr Tyr Phe Tyr Trp Tyr Leu Xaa3 Lys Pro Gly Gln Pro Pro Gln Leu Leu Ile Tyr Gln Met Ser Asn Leu Ala Ser Gly Val Pro Asp Arg Phe Ser Gly Ser Gly Ser Gly Thr Asp Phe Thr Leu Lys Ile Ser Arg Val Glu Ala Glu Asp Val Gly Val Tyr Tyr Cys Ala Gln Asn Leu Glu Leu Pro Leu Thr Phe Gly Gly Gly Thr Lys Val Glu Ile Lys (SEQ ID NO: 40), or at least the CDRs thereof, wherein (a) Xaa1 is serine (Ser) or arginine (Arg), (b) Xaa2 is glycine (Gly) or alanine (Ala), and (c) Xaa3 is glutamine (Gln) or histidine (His).

The light chain variable region can comprise, consist of, or consist essentially of the amino acid sequence of SEQ ID NO: 40, or at least the CDRs thereof, with the aforementioned amino acid substitutions in any combination. In one embodiment, the immunoglobulin light chain polypeptide comprises, consists of, or consists essentially of an amino acid sequence of any one of SEQ ID NO: 41, SEQ ID NO: 42, SEQ ID NO: 43, or SEQ ID NO: 44; or at least the CDRs thereof.

In some embodiments, the light chain variable region comprises a LCDR1 comprising, consisting of, or consisting essentially of the amino acid sequence (a) Arg Ser Ser Lys Ser Leu Leu His Ser Asn Gly Ile Thr Tyr Phe Tyr (SEQ ID NO: 81); (b) Arg Ser Ser Lys Ser Leu Leu His Ser Asn Ala Ile Thr Tyr Phe Tyr (SEQ ID NO: 82); or (c) Arg Ser Ser Lys Ser Leu Leu His Arg Asn Ala Ile Thr Tyr Phe Tyr (SEQ ID NO: 83); a LCDR2 comprising, consisting of, or consisting essentially of the amino acid sequence Gln Met Ser Asn Leu Ala Ser (SEQ ID NO: 84); and a LCDR3 comprising, consisting of, or consisting essentially of the amino acid sequence Ala Gln Asn Leu Glu Leu Pro Leu Thr (SEQ ID NO: 85).

In further embodiments, the immunoglobulin light chain variable region comprises, consists of, or consists essentially of the amino acid sequence of Asp Ile Gln Met Thr Gln Ser Pro Ser Ser Leu Ser Ala Ser Val Gly Asp Arg Val Thr Ile Thr Cys Arg Ala Ser Gln Xaa1 Ile Asn Asn Tyr Leu Asn Trp Tyr Gln Gln Lys Pro Gly Lys Ala Pro Lys Leu Leu Ile Tyr Tyr Thr Ser Xaa2 Leu His Ser Gly Val Pro Ser Arg Phe Ser Xaa3 Ser Gly Ser Gly Xaa4 Asp Xaa5 Thr Phe Thr Ile Ser Ser Leu Gln Pro Glu Asp Ile Ala Thr Tyr Tyr Cys Gln Gln Gly His Thr Leu Pro Trp Thr Phe Gly Gly Gly Thr Lys Val Glu Ile Lys Xaa6 Xaa7 (SEQ ID NO: 58), or at least the CDRs thereof, wherein (a) Xaa1 is aspartic acid (Asp) or tryptophan (Trp), (b) Xaa2 is arginine (Arg) or methionine (Met), (c) Xaa3 is glycine (Gly), serine (Ser) or proline (Pro), (d) Xaa4 is threonine (Thr) or asparagines (Asn), (e) Xaa5 is phenylalanine (Phe) or tyrosine (Tyr), (f) Xaa6 is arginine (Arg) or absent, and (g) Xaa7 is threonine (Thr) or absent. In some embodiments, the immunoglobulin light chain variable region comprises, consists of, or consists essentially of the amino acid sequence Asp Ile Gln Met Thr Gln Ser Pro Ser Ser Leu Ser Ala Ser Val Gly Asp Arg Val Thr Ile Thr Cys Arg Ala Ser Gln Asp Ile Asn Asn Tyr Leu Asn Trp Tyr Gln Gln Lys Pro Gly Lys Ala Pro Lys Leu Leu Ile Tyr Tyr Thr Ser Arg Leu His Ser Gly Val Pro Ser Arg Phe Ser Xaa1 Ser Gly Ser Gly Thr Asp Xaa2 Thr Phe Thr Ile Ser Ser Leu Gln Pro Glu Asp Ile Ala Thr Tyr Tyr Cys Gln Gln Gly His Thr Leu Pro Trp Thr Phe Gly Gly Gly Thr Lys Val Glu Ile Lys (SEQ ID NO: 45), or at least the CDRs thereof, wherein (a) Xaa1 is serine (Ser) or proline (Pro), and (b) Xaa2 is phenylalanine (Phe) or tyrosine (Tyr).

The light chain variable region can comprise, consist of, or consist essentially of the amino acid sequence of SEQ ID NO: 58 or SEQ ID NO: 45, or at least the CDRs thereof, with one or more of the aforementioned amino acid substitutions in any suitable combination. In one embodiment, the immunoglobulin light chain polypeptide comprises, consists of, or consists essentially of an amino acid sequence of any one of SEQ ID NO: 46, SEQ ID NO: 47, or SEQ ID NO: 55, or at least the CDRs thereof.

In some embodiments, the light chain variable region comprises an LCDR1 comprising, consisting of, or consisting essentially of the amino acid sequence (a) Arg Ala Ser Gln Asp Ile Asn Asn Tyr Leu Asn (SEQ ID NO: 86); or (b) Arg Ala Ser Gln Trp Ile Asn Asn Tyr Leu Asn (SEQ ID NO: 87); an LCDR2 comprising, consisting of, or consisting essentially of an amino acid sequence (a) Tyr Thr Ser Arg Leu His Ser (SEQ ID NO: 88); or (b) Tyr Thr Ser Met Leu His Ser (SEQ ID NO: 89); and an LCDR3 comprising, consisting of, or consisting essentially of the amino acid sequence Gln Gln Gly His Thr Leu Pro Trp Thr (SEQ ID NO: 90).

In another embodiment, the immunoglobulin light chain variable region comprises, consists of, or consists essentially of the amino acid sequence of SEQ ID NO: 48, SEQ ID NO: 49, or SEQ ID NO: 50, or at least the CDRs thereof.

In some embodiments, the immunoglobulin light chain variable region comprises an amino acid sequence that is at least 90% identical (e.g., at least 91%, at least 92%, at least 93%, at least 94%, at least 95%, at least 96%, at least 97%, at least 98%, at least 99%, or 100% identical) to any of the foregoing immunoglobulin light chain variable region sequences. Nucleic acid or amino acid sequence “identity” can be determined using the methods described herein.

As described above, the IL-36R antibody or antibody fragment can comprise an immunoglobulin heavy chain variable region and light chain variable region have any of the foregoing heavy and light chain variable region sequences, or the CDRs thereof. The CDR sequences can be the CDR sequences set forth herein or the CDR sequences as determined using any of several known methods (e.g., Kabat, Chothia, Martin (Enhanced Chothia), IGMT, or AHo).

In one embodiment, the IL-36R antibody or antibody fragment has an immunoglobulin heavy chain variable region comprising SEQ ID NO: 15 or SEQ ID NO: 22, or at least the CDR regions thereof; and an immunoglobulin light chain variable region comprising SEQ ID NO: 40 or SEQ ID NO: 44, or at least the CDR sequences thereof, wherein the CDRs are as determined with determined in accordance with any of the various known immunoglobulin numbering schemes, particularly in accordance with Kabat, Chothia, Martin (Enhanced Chothia), IGMT, or AHo. For example, in some embodiments, the antibody or antibody fragment comprises a heavy chain variable region of SEQ ID NO: 22 and light chain variable region of SEQ ID NO: 44, or at least the CDRs thereof as determined by Kabat. In some embodiments, the antibody or antibody fragment comprises a heavy chain variable region of SEQ ID NO: 22 and light chain variable region of SEQ ID NO: 44, or at least the CDRs thereof as determined by Chothia. In some embodiments, the antibody or antibody fragment comprises a heavy chain variable region of SEQ ID NO: 22 and light chain variable region of SEQ ID NO: 44, or at least the CDRs thereof as determined by Martin. In some embodiments, the antibody or antibody fragment comprises a heavy chain variable region of SEQ ID NO: 22 and light chain variable region of SEQ ID NO: 44, or at least the CDRs thereof as determined by IGMT. In some embodiments, the antibody or antibody fragment comprises a heavy chain variable region of SEQ ID NO: 22 and light chain variable region of SEQ ID NO: 44, or at least the CDRs thereof as determined by AHo.

In some embodiments, the IL-36R antibody or antibody fragment comprises a heavy chain variable region comprising: an HCDR1 comprising, consisting of, or consisting essentially of the amino acid sequence selected from the group consisting of (a) Tyr Thr Phe Thr Asn Tyr Trp Met His (SEQ ID NO: 64); (b) Tyr Thr Phe Thr Asn Tyr Trp Met Asn (SEQ ID NO: 65); (c) Tyr Thr Phe Thr Asn Tyr Trp Met Tyr (SEQ ID NO: 66); and (d) Tyr Thr Phe Thr Asn Tyr Tyr Met Asn (SEQ ID NO: 67); an HCDR2 comprising, consisting of, or consisting essentially of the amino acid sequence selected from the group consisting of (a) Met Phe Asp Pro Ser Asn Ser Val Thr Arg Leu Asn Gln Lys Phe Lys Asp (SEQ ID NO: 68); (b) Met Phe Glu Pro Ser Asn Ala Val Thr Arg Leu Asn Gln Lys Phe Lys Asp (SEQ ID NO: 69); (c) Met Phe His Pro Ser Asn Ala Val Thr Arg Leu Asn Gln Lys Phe Lys Asp (SEQ ID NO: 70); and (d) Met Phe His Pro Thr Gly Asp Val Thr Arg Leu Asn Gln Lys Phe Lys Asp (SEQ ID NO: 71); and/or an HCDR3 comprising, consisting of, or consisting essentially of the amino acid sequence Thr Thr Ser Met Ile Ile Gly Gly Phe Ala Tyr (SEQ ID NO: 72); and comprises a light chain variable region comprising a LCDR1 comprising, consisting of, or consisting essentially of the amino acid sequence (a) Arg Ser Ser Lys Ser Leu Leu His Ser Asn Gly Ile Thr Tyr Phe Tyr (SEQ ID NO: 81); (b) Arg Ser Ser Lys Ser Leu Leu His Ser Asn Ala Ile Thr Tyr Phe Tyr (SEQ ID NO: 82); or (c) Arg Ser Ser Lys Ser Leu Leu His Arg Asn Ala Ile Thr Tyr Phe Tyr (SEQ ID NO: 83); a LCDR2 comprising, consisting of, or consisting essentially of the amino acid sequence Gln Met Ser Asn Leu Ala Ser (SEQ ID NO: 84); and a LCDR3 comprising, consisting of, or consisting essentially of the amino acid sequence Ala Gln Asn Leu Glu Leu Pro Leu Thr (SEQ ID NO: 85).

In a particular embodiment, the IL-36R antibody or antibody fragment comprises a heavy chain variable region comprising: an HCDR1 comprising, consisting of, or consisting essentially of the amino acid sequence selected from the group consisting of Tyr Thr Phe Thr Asn Tyr Trp Met Asn (SEQ ID NO: 65); an HCDR2 comprising, consisting of, or consisting essentially of the amino acid sequence selected from the group consisting of Met Phe His Pro Thr Gly Asp Val Thr Arg Leu Asn Gln Lys Phe Lys Asp (SEQ ID NO: 71); and/or an HCDR3 comprising, consisting of, or consisting essentially of the amino acid sequence Thr Thr Ser Met Ile Ile Gly Gly Phe Ala Tyr (SEQ ID NO: 72); and comprises a light chain variable region comprising a LCDR1 comprising, consisting of, or consisting essentially of the amino acid sequence Arg Ser Ser Lys Ser Leu Leu His Arg Asn Ala Ile Thr Tyr Phe Tyr (SEQ ID NO: 83); a LCDR2 comprising, consisting of, or consisting essentially of the amino acid sequence Gln Met Ser Asn Leu Ala Ser (SEQ ID NO: 84); and a LCDR3 comprising, consisting of, or consisting essentially of the amino acid sequence Ala Gln Asn Leu Glu Leu Pro Leu Thr (SEQ ID NO: 85).

Furthermore, the IL-36R antibody or antibody fragment can comprise an immunoglobulin heavy chain variable region and light chain variable region having specified percent identities to the heavy and light chain variable region sequences, such as at least 90% identical (e.g., at least 91%, at least 92%, at least 93%, at least 94%, at least 95%, at least 96%, at least 97%, at least 98%, at least 99%, or 100% identical). In some embodiments, the variance in sequence occurs outside the CDRs (as determined by any known method including Kabat, Chothia, Martin (Enhanced Chothia), IGMT, or AHo), such that the heavy and light chain sequences having the specified sequence identity to the specific sequences set forth herein retain the CDRs of such sequences. In an embodiment, the IL-36R antibody or antibody fragment comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 15 or SEQ ID NO: 22, optionally wherein the sequence retains the CDRs of SEQ ID NO: 15 or SEQ ID NO: 22; and comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 40 or SEQ ID NO: 44, optionally wherein the sequence retains the CDRs of SEQ ID NO: 40 or SEQ ID NO: 44; wherein the CDRs are as determined in accordance with any of the various known immunoglobulin numbering schemes, particularly in accordance with Kabat, Chothia, Martin (Enhanced Chothia), IGMT, or AHo. In a particular embodiment, the IL-36R antibody or antibody fragment comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 22, optionally wherein the sequence retains the CDRs of SEQ ID NO: 22; and comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 44, optionally wherein the sequence retains the CDRs of SEQ ID NO: 44; wherein the CDRs are as determined in accordance with any of the various known immunoglobulin numbering schemes, particularly in accordance with Kabat, Chothia, Martin (Enhanced Chothia), IGMT, or AHo.

Variation in sequence identity can be accomplished through addition, substitution, or deletion of one or more amino acid residues. An amino acid “replacement” or “substitution” refers to the replacement of one amino acid at a given position or residue by another amino acid at the same position or residue within a polypeptide sequence. The amino acid replacement or substitution can be conservative, semi-conservative, or non-conservative depending upon whether the substitution is by an amino acid residue that has similar properties to the residue being replaced. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids can be broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed herein, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

When the immunoglobulin light chain or heavy chain variable region “consists essentially of” any of the foregoing heavy or light chain variable region amino acid sequences, additional components can be included in the polypeptide that do not materially affect the polypeptide, such as those described herein. When the immunoglobulin light chain or heavy chain variable region “consists of”, the polypeptide does not comprise any additional components.

The IL-36R antibody or antibody fragment can be a binding agent that competes with an IL-36R binding agent comprising an immunoglobulin heavy chain polypeptide or light chain polypeptide described herein for binding to IL-36R, e.g., binds to the same epitope or an overlapping epitope. Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

The amount of antibody, or antigen-binding fragment thereof, contained within the pharmaceutical formulations of the present invention may vary depending on the specific properties desired of the formulations, as well as the particular circumstances and purposes for which the formulations are intended to be used. In certain embodiments the pharmaceutical formulations are liquid formulations that main contain the antibody or antigen-binding fragment thereof in an amount from about 75 mg/mL to about 175 mg/mL (e.g., about 75 mg/mL, about 85 mg/mL, about 95 mg/mL, about 105 mg/mL, about 115 mg/mL, about 125 mg/mL, about 135 mg/mL, about 145 mg/mL, about 155 mg/mL, about 165 mg/mL, about 175 mg/mL, or a range defined by any two of the foregoing values), preferably from about 75 mg/mL to about 150 mg/mL (e.g., about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, or a range defined by any two of the foregoing values), and more preferably from about 75 mg/mL to about 125 mg/mL.

### Method of Treatment

Provided herein is a method of treating a subject in need of an anti-IL-36R antibody or antigen binding antibody fragment, the method comprising administering an effective amount of the pharmaceutical formulation described herein. The subject can be any subject in need of an anti-IL-36R antibody or antigen binding antibody fragment, including any subject that has a disorder condition that is responsive to IL-36R inhibition or neutralization. A disorder that is “responsive to IL-36R inhibition” or “responsive to IL-36R neutralization” refers to any disease or disorder in which a decrease in IL-36R levels or activity has a therapeutic benefit in mammals, preferably humans, or the improper expression (e.g., overexpression) or increased activity of IL-36R causes or contributes to the pathological effects of the disease or disorder. Disorders that are responsive to IL-36R inhibition include, for example, inflammatory diseases, autoimmune diseases, respiratory diseases, metabolic disorders, and cancer.

Inflammatory disorders include, for example, allergic inflammation of the skin, lungs, and gastrointestinal tract, atopic dermatitis (also known as atopic eczema), asthma (allergic and non-allergic), epithelial-mediated inflammation, hidradenitis suppurativa, acne, fibrosis (e.g., idiopathic pulmonary fibrosis, scleroderma, kidney fibrosis, and scarring), allergic rhinitis, food allergies (e.g., allergies to peanuts, eggs, dairy, shellfish, tree nuts, etc.), seasonal allergies, and other allergies.

The inventive method can be used to treat any type of autoimmune disease (i.e., as disease or disorder caused by immune system overactivity in which the body attacks and damages its own tissues), such as those described in, for example, Mackay I. R. and Rose N. R., eds., The Autoimmune Diseases, Fifth Edition, Academic Press, Waltham, MA (2014). Examples of autoimmune diseases that can be treated by the inventive method include, but are not limited to, multiple sclerosis, asthma, type 1 diabetes mellitus, rheumatoid arthritis, scleroderma, Crohn's disease, psoriasis vulgaris (commonly referred to as psoriasis), pustular psoriasis, generalized pustular psoriasis (GPP), palmo-plantar pustulosis (PPP), inflammatory bowel disease, psoriatic arthritis, multiple sclerosis, rheumatoid arthritis, systemic lupus erythematosus (SLE), ulcerative colitis, ankylosing spondylitis, ichthyosis, and skin toxicity. In a preferred embodiment, the inventive method is used to treat pustular psoriasis, generalized pustular psoriasis, palmo-plantar pustulosis (PPP), or psoriasis vulgaris.

Pustular psoriasis is a rare form of psoriasis characterized by white pustules surrounded by red skin. Generalized pustular psoriasis (GPP) is a life-threatening disease characterized by sudden, repeated episodes of high-grade fever, generalized rash, and disseminated pustules, with hyperleukocytosis and elevated serum levels of C-reactive protein, which can be caused by a deficiency in the interleukin-36-receptor antagonist (interleukin-36Ra) (Marrakchi et al., N. Engl. J. Med., 365(7):620-628 (2011)). GPP often presents in patients with existing or prior psoriasis vulgaris (PV); however, GPP can develop in patients without a history of PV (Sugiura et al., J. Invest. Derm., 133: 2514-2521 (2013)). Palmo-plantar pustulosis is a chronic inflammatory skin disease characterized by sterile pustules and red, scaly skin on the palms and soles that considerably impairs the quality of life of affected individuals (de Waal, A. C. and van de Kerkhof, P. C. M., J. Dermatological Treatment, 22(2): 102-105 (2011)).

Examples of respiratory diseases that can be treated by the inventive method include, but are not limited to, asthma, cystic fibrosis, emphysema, chronic obstructive pulmonary disease (COPD), and acute respiratory distress syndrome. Examples of metabolic disorders that can be treated by the inventive method include, but are not limited to, obesity, type 2 diabetes, atherosclerosis, and cardiovascular disease.

The inventive method can be used to treat any type of cancer known in the art, including but not limited to, melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, leukemia, lymphoma, and Merkel cell carcinoma (see, e.g., Bhatia et al., Curr. Oncol. Rep., 13(6): 488-497 (2011)).

Administration of the pharmaceutical formulation described herein induces an immune response in a mammal. An “immune response” can entail, for example, antibody production and/or the activation of immune effector cells (e.g., T-cells).

As used herein, the terms “treatment,” “treating,” and the like refer to obtaining a desired pharmacologic and/or physiologic effect. Preferably, the effect is therapeutic, i.e., the treatment reduces the severity of one or more adverse symptoms of a patient in need of an anti-IL-36R antibody, including, but not limited to, scale removal, erythema, inhibition of pruritus, and reduced inflammation.

To this end, the inventive method comprises administering a “therapeutically effective amount” of the pharmaceutical formulation described herein.

A “therapeutically effective amount” refers to an amount effective, at dosages and for periods of time necessary, to achieve a desired pharmacologic and/or physiologic effect. The therapeutically effective amount may vary according to factors such as the disease state, age, sex, and weight of the individual, and the ability of the pharmaceutical formulation to elicit a desired response in the individual. For example, a therapeutically effective amount of the pharmaceutical formulation described herein is an amount that decreases the bioactivity of any one of the IL-36 cytokines and/or IL-36R, such that a therapeutically effective amount of the pharmaceutical formulation described herein is an amount that decreases IL-36 bioactivity in a subject. In another example, a therapeutically effective amount of the pharmaceutical formulation described herein is an amount that decreases the adverse symptoms of the subject.

In some embodiments, the pharmacologic and/or physiologic effect may be prophylactic, i.e., the effect completely or partially prevents the disease, disorder, or condition characterized by increased IL-36 bioactivity or symptom thereof. In this respect, the inventive method comprises administering a “prophylactically effective amount” of the pharmaceutical formulation. A “prophylactically effective amount” refers to an amount effective, at dosages and for periods of time necessary, to achieve a desired prophylactic result. In some embodiments, the subject can be a subject with a genetic predisposition to a disease, disorder, or condition characterized by increased IL-36 bioactivity.

The following examples further illustrate the invention but, of course, should not be construed as in any way limiting its scope.

### Example 1

This Example describes the sample preparation of four different formulations of IL-36R antibody formulations.

Starting with about 200 mL of the UF/DF rinse (lot C6903), an anti-IL-36R antibody (heavy chain variable region SEQ ID NO: 22 and light chain variable region SEQ ID NO: 44) was buffer exchanged into formulation F02A, F02B, F02C (described below in Table 1) by UF/DF. Diafiltration was performed by 200 cm² Sartorius vivaflow TFF cassette. About seven (7) diavolumes (˜150 mL/DV) was achieved for complete buffer exchange. The solutions were then concentrated to about 30-35 mL to achieve protein concentration greater than 100 mg/mL and diluted with UF/DF rinse as needed to achieve target concentration of 100 mg/mL. A sample of F01 was also prepared by concentrating the UF/DF rinse to a target concentration of 100 mg/mL. For all samples, 1 mL of each solution was put into glass drug Product (DP) vials with rubber stopper, sealed with parafilm, and stored at 2-8° C. until placed at 45° as described in the next example.

### Example 2

This example demonstrates the relative protein concentration, pH osmolality and viscosity of four formulations.

A formulation verification study was initiated at AnaptysBio (San Diego, CA). For four different formulations, protein concentration (mg/mL), pH, osmolality (mOsm/kg), and viscosity (cP) were measured.

pH measurement was performed using an Oakton PC700 pH meter with a Mettler Toledo inLab micro probe, 3 mol/L KCl. Instrument control and data acquisition were performed with the meter itself. pH values were recorded manually from the meter display once pH stabilization was achieved. The pH procedure was as follows: 200 μL of each dosing solution was pipetted into a 1.5 mL Eppendorf tube for pH measurement. pH measurements were recorded once the meter reached stabilization.

Osmolality analysis was performed using a Precision Systems Osmette III-5010 auto osmometer. Instrument control and data acquisition were performed with the meter itself. Osmolality values were recorded manually from the meter display once instrument stabilization was achieved. The Osmolality procedure was as follows: 10 μL in triplicate of each dosing solution was pipetted into the instrument for measurement. Osmolality measurements were recorded once the instrument reached stabilization.

Viscosity analysis was performed using a RheoSense HVROC-L (microVISC) viscometer. Instrument control and data acquisition were performed using the micro VISC software. For all measurements, the instrument was placed inside the microVISC temperature control chamber, which was set to 25° C. The viscosity procedure was as follows: 400 μL of each formulated sample at 100 mg/mL was drawn into microVISC single use pipettes for auto-inject by the instrument for each measurement. Al sample injections were performed in at least triplicate if possible. Cleanings were performed with injections of 1% Aquet detergent solution between each sample set. Viscosity standards were also checked to verify the integrity of the viscosity data collected. Viscosity data captured by the microVISC software were exported to Microsoft excel. Table 1 shows the results.

TABLE 1

| | | Protein | | | Measured |
|---|---|---|---|---|---|
| Formulation | Formulation | Concentration | Osmolality | Measured | Viscosity |
| # | Composition | (mg/mL) | (mOsm/kg) | pH | (cP) |
| F01 | 25 mM histidine 145 | 100.0 | 323 | 6.00 | 4.027 ± |
| | mM sorbitol 60 mM | | | | 0.013 |
| | NaCl 0.02% PS20 pH | | | | |
| | 6.0 | | | | |
| F02A | 10 mM histidine 280 | 101.5 | 378 | 6.17 | 9.585 ± |
| | mM proline 0.03% | | | | 0.047 |
| | PS20 pH 6.0 | | | | |
| F02B | 10 mM histidine 70 | 100.4 | 335 | 6.21 | 12.454 ± |
| | mM sorbitol 210 mM | | | | 0.077 |
| | proline 0.03% PS20 pH | | | | |
| | 6.0 | | | | |
| F02C | 25 mM histidine 280 | 101.2 | 373 | 5.88 | 7.261 ± |
| | mM sorbitol 0.05% | | | | 0.013 |
| | PS30 pH 5.8 | | | | |

Measured osmolality of all formulations were higher than expected. This is likely attributed to the Donon effect typically observed when using a centrifuge device to concentrate a protein solution. Osmolality standards that were used to ensure proper performance of the osmometer were all within acceptable range. A difference in measured viscosity (cP) was also observed for the four formulations with F02C being closest to F01 and F02B being the highest.

### Example 3

This Experiment demonstrates the increased stability of the F02A formulation.

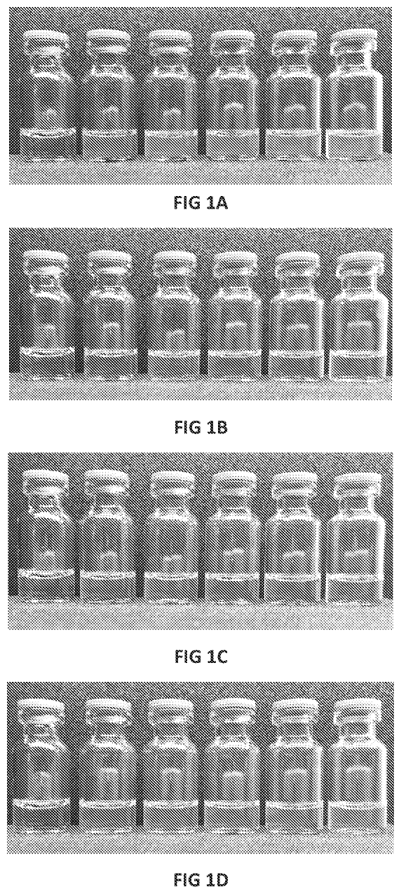

IL-36R antibody in four different formulations (i.e., F01, F02A, F02B, F02C, as described in Table 1) were incubated at 45° ° C. for up to 8 days. Color, clarity, and visual appearance of 1 mL samples were evaluated in glass DP vials against appropriate standards. Sample images were captured within a light box under illumination against a black background. FIG. 1A shows images of the visual appearance of F01 at 0, 5, 6, 7, and 8 days as compared to water. FIG. 1B shows images of the visual appearance of F02A at 0, 5, 6, 7, and 8 days as compared to water. FIG. 1C shows images of the visual appearance of F02B at 0, 5, 6, 7, and 8 days as compared to water. FIG. 1D shows images of the visual appearance of F02C at 0, 5, 6, 7, and 8 days as compared to water. Comparatively, F02A, F02B, and F02C were substantially less opalescent than F01. Opalescence observed for F02C was similar to F01. F02A and F02B were noticeably less opalescent than F02C. The level of opalescence did not seem to increase in any of the formulations after being stressed at 45° C. for 8 days.

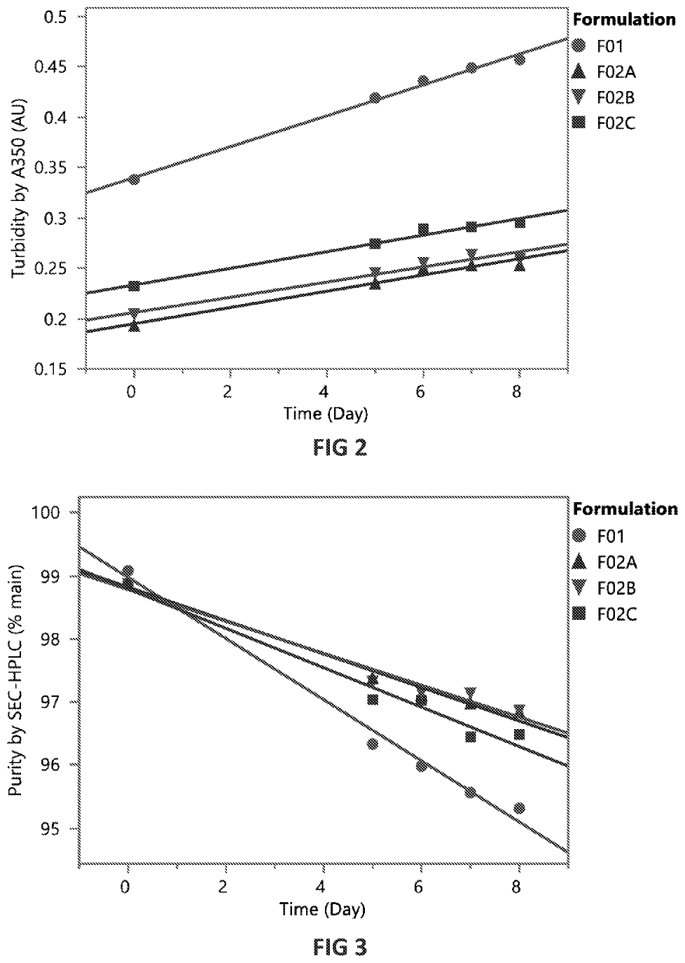

Following storage at 45° C. for 8 days, each sample was measured for turbidity by A350. Turbidity analysis was performed using a Thermo Scientific Nanodrop 2000c spectrophotometer. Instrument control and data acquisition were performed using the nanodrop software. The turbidity procedure was as follows: 2 μL of each formulated sample at 100 mg/mL and controls were applied to the nanodrop for each measurement in the UV-VIS application using a 10 mm path length recording sample absorbance at 350 nm. All measurements were performed in at least triplicate. NTU turbidity standards were also checked to reference the sample turbidity data collected. Turbidity data captured by the nanodrop software were exported to Microsoft excel. Table 2 shows the results of turbidity by A350. FIG. 2 shows a bivariate plot of turbidity by A350 of IL-36R formulations stored at 45° C. Table 3 shows the goodness of fit: linear model for turbidity (A350) by Time (Day).

TABLE 2
Stressed Study results: Turbidity by A₃₅₀(AU)

| Formulation | Time (Day) | | | | |
|---|---|---|---|---|---|
| # | 0 | 5 | 6 | 7 | 8 |
| F01 | 0.338 | 0.419 | 0.436 | 0.449 | 0.457 |
| F02A | 0.193 | 0.235 | 0.250 | 0.253 | 0.253 |
| F02B | 0.204 | 0.245 | 0.255 | 0.263 | 0.259 |
| F02C | 0.232 | 0.274 | 0.289 | 0.291 | 0.295 |

TABLE 3
Goodness of Fit: Linear Model for Turbidity (A₃₅₀) by Time (Day)

| Formulation # | Slope (AU/day) | R-Squared | p-Value |
|---|---|---|---|
| F01 | 0.015 | 0.9934 | 0.0002 |
| F02A | 0.008 | 0.9647 | 0.0028 |
| F02B | 0.007 | 0.9604 | 0.0034 |
| F02C | 0.008 | 0.9781 | 0.0014 |

All formulations showed an increasing trend in turbidity as indicated by the p-values reported in Table 3. To further evaluate the turbidity data, a common slopes test was used to compare the changes in turbidity between the four formulations. The analysis was performed using a crossed design in Fit Model platform in JMP® to examine if there is a statistically significant difference between the slopes of each formulation. The test demonstrated that there is a statistically significant difference in change of turbidity over time between all formulations as indicated as an F ration greater than 4 and a p-value that is <0.0001. F01 is inferior to F02A-C as it has a slope that is almost twice as high. The results of the common slopes test are shown in in Table 4

TABLE 4
Common Slopes Analysis: Turbidity (A₃₅₀) by Time (Day)

| | | | Sum of | | |
|---|---|---|---|---|---|
| Source | Nparm | DF | Squares | F Ratio | Prob > F |
| Formulation | 3 | 3 | 0.10897380 | 1438.378 | <.0001* |
| Time (Day) | 1 | 1 | 0.01490619 | 590.2539 | <.0001* |
| Formulation*Time | 3 | 3 | 0.00159796 | 21.0920 | <.0001* |
| (Day) | | | | | |

The samples that were stored at 45° C. for up to 8 days were measured for purity by SEC-HPLC (% main). Sample purity by SEC-HPLC was determined using a Tosoh Tskgel G3000SWx1 5 μm column. Samples were analyzed after dilution to 10 mg/mL in formulation buffer (F01, F02A, F02B, F02C) with an injection volume of 10 μL, a run time of 20 min, a flow rate of 1.0 mL/min, and a column temperature of 25° C. Samples were analyzed in singlicate. Table 5 shows the results of the purity measurements. FIG. 3 shows a bivariate plot of SEC-HPLC (% main) of IL-36R formulations stored at 45° C. Table 6 shows the results of a goodness of fit analysis of the results. All formulations showed a decreasing trend in purity as indicated by the p-values reported in Table 6.

TABLE 5
Purity by SEC-HPLC (% main)

| Formulation | Time (Day) | | | | |
|---|---|---|---|---|---|
| # | 0 | 5 | 6 | 7 | 8 |
| F01 | 99.08 | 96.33 | 95.98 | 95.56 | 95.31 |
| F02A | 98.89 | 97.38 | 97.11 | 96.98 | 96.84 |
| F02B | 98.87 | 97.32 | 97.15 | 97.13 | 96.86 |
| F02C | 98.86 | 97.03 | 97.02 | 96.44 | 96.48 |

TABLE 6
Goodness of Fit: Linear Model for
SEC-HPLC (% main) by Time (Day)

| Formulation # | Slope (% main/day) | R-Squared | p-Value |
|---|---|---|---|
| F01 | −0.485 | 0.9877 | 0.0006 |
| F02A | −0.265 | 0.9812 | 0.0011 |
| F02B | −0.253 | 0.9673 | 0.0025 |
| F02C | −0.313 | 0.9699 | 0.0022 |

A common slopes test was used to compare the change in purity between the four formulations. The results of the common slopes test are shown in Table 7. The analysis was performed using a crossed design in Fit Model platform in JMP® to examine if there is a statistically significant difference between the slopes of each formulation. The test demonstrated that there is a statistically significant difference in change of purity over time between all formulations as indicated by an F ratio greater than 4 and a p-value of 0.003. As shown in Table 7, F01 and F02C is inferior to F02A and F02B as indicated by a slope that is almost twice as high. The similar decrease in purity observed for F02C is reasonable as the composition of this formulation is similar to that of F01.

TABLE 7
Common Slopes Analysis: SEC-HPLC (% main) by Time (Day)

| | | | Sum of | | |
|---|---|---|---|---|---|
| Source | Nparm | DF | Squares | F Ratio | Prob > F |
| Formulation | 3 | 3 | 3.349860 | 36.4974 | <.0001* |
| Time (Day) | 1 | 1 | 16.807690 | 549.3689 | <.0001* |
| Formulation*Time | 3 | 3 | 1.335296 | 14.5483 | 0.0003* |
| (Day) | | | | | |

This Experiment shows that F02A and F02B experience greater stability in terms of turbidity and purity over time when exposing the formulations to 45° C. for 8 days.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A stable, aqueous pharmaceutical formulation comprising (a) water, (b) a histidine buffer, (b) a stabilizer comprising proline and/or sorbitol, (c) a non-ionic surfactant, and (d) an antibody or antigen-binding antibody fragment.

2. The pharmaceutical formulation of claim 1, wherein the stabilizer comprises proline.

3. The pharmaceutical formulation of claim 2, wherein the pharmaceutical formulation comprises about 100 to 300 mM proline.

4. (canceled)

5. The pharmaceutical formulation of claim 1, wherein the stabilizer comprises sorbitol.

6. The pharmaceutical formulation of claim 5, wherein the pharmaceutical formulation comprises about 100 to 300 mM sorbitol.

7. (canceled)

8. The pharmaceutical formulation of claim 1, wherein the stabilizer comprises proline and sorbitol.

9. The pharmaceutical formulation of claim 8, wherein the pharmaceutical formulation comprises about 100 to 300 mM combined proline and sorbitol.

10. (canceled)

11. The pharmaceutical formulation of claim 8, wherein the pharmaceutical formulation comprises about 50-80 mM sorbitol and 180-250 mM proline.

12. The pharmaceutical formulation of claim 1, wherein the pharmaceutical formulation comprises about 5-35 mM histidine.

13. (canceled)

14. The pharmaceutical formulation of claim 1, wherein the pH is about 5.0-6.5.

15. (canceled)

16. The pharmaceutical formulation of claim 1, wherein the non-ionic surfactant is a polysorbate.

17. The pharmaceutical formulation of claim 16, wherein the nonionic surfactant is a polysorbate-20.

18. The pharmaceutical formulation of claim 17, wherein the pharmaceutical formulation comprises about 0.01 to 0.1 wt. % polysorbate-20.

19. (canceled)

20. The pharmaceutical formulation of claim 1, wherein the pharmaceutical formulation comprises about 75-175 mg/mL of an antibody or antigen-binding antibody fragment.

21. (canceled)

22. The pharmaceutical formulation of claim 1, wherein the pharmaceutical formulation has a viscosity of less than 15 cps.

23. The pharmaceutical formulation of claim 1, wherein the pharmaceutical formulation comprises less than 10 mM NaCl.

24. The pharmaceutical formulation of claim 1, wherein the formulation comprises:
(a) about 75-150 mg/mL antibody or antigen binding antibody fragment;
(b) about 9-11 mM histidine;
(c) about 275-285 mM proline; and
(d) about 0.01%-0.05% polysorbate-20;
or
(a) about 75-150 mg/mL antibody or antigen binding antibody fragment;
(b) about 9-11 mM histidine;
(c) about 205-215 mM proline;
(d) about 0.01%-0.05% polysorbate-20; and
(e) about 65-75 mM sorbitol;
or
(a) about 75-150 mg/mL antibody or antigen binding antibody fragment;
(b) about 24-26 mM histidine;
(c) about 275-285 mM proline; and
(d) about 0.03%-0.07% polysorbate-20.

25. The pharmaceutical formulation of claim 1, wherein the formulation comprises:
(a) about 75-150 mg/mL antibody or antigen binding antibody fragment;
(b) about 10 mM histidine;
(c) about 280 mM proline; and
(d) about 0.03% polysorbate-20;
or
(a) about 75-150 mg/mL antibody or antigen binding antibody fragment;
(b) about 10 mM histidine;
(c) about 210 mM proline;
(d) about 0.03% polysorbate-20; and
(e) about 70 mM sorbitol;
or
(a) about 75-150 mg/mL antibody or antigen binding antibody fragment;
(b) about 25 mM histidine;
(c) about 280 mM proline; and
(d) about 0.05% polysorbate-20.

26. The pharmaceutical formulation of claim 1, wherein the antibody is an anti-IL-36 antibody, optionally comprising
(i) a light chain variable region comprising a complementarity determining region (CDR) 1 domain (CDRL1) comprising the amino acid sequence of SEQ ID NO: 83; a CDRL2 domain comprising the amino acid sequence of SEQ ID NO: 84; and a CDRL3 domain comprising the amino acid sequence of SEQ ID NO: 85, and
(ii) a heavy chain variable region comprising a CDHR1 domain comprising the amino acid sequence of SEQ ID NO: 65: a CDRH2 domain comprising the amino acid sequence of SEQ ID NO: 71; and a CDRH3 domain comprising the amino acid sequence of SEQ ID NO: 72.

27. The pharmaceutical formulation of claim 26, wherein the formulation comprises an anti-IL-36R antibody comprising a light chain variable region comprising the amino acid sequence of SEQ ID NO: 44 and a heavy chain variable region comprising the amino acid sequence of SEQ ID NO: 22.

28. A method of treating a patient in need of an anti-IL-36R antibody, the method comprising administering an effective amount of the pharmaceutical formulation of claim 26.

29. The method of claim 28 for treating an autoimmune, inflammatory, respiratory, or metabolic disease or disorder, or cancer.

30. (canceled)
